(19) United States
(12) Patent Application Publication
(10) Pub. No.: US 2026/0284223 A1
(43) Pub. Date: Sep. 24, 2026

(54) PREPARATION OF SOLID CYCLODEXTRIN COMPLEXES FOR OPHTHALMIC ACTIVE PHARMACEUTICAL INGREDIENT DELIVERY

(71) Applicant: Oculis Operations Sàrl, Lausanne (CH)

(72) Inventors: Thorsteinn Loftsson, Reykjavik (IS); Einar Stefansson, Reykjavik (IS)

(73) Assignee: Oculis Operations Sàrl, Lausanne (CH)

(21) Appl. No.: 19/363,578

(22) Filed: Oct. 20, 2025

Related U.S. Application Data
(63) Continuation of application No. 18/014,060, filed on Jun. 30, 2021, now abandoned.

(30) Foreign Application Priority Data
Jun. 30, 2020 (EP) 20183307.6

(51) Int. Cl. A61K 47/69; A61K 9/00; A61K 47/10; A61K 47/18

(52) U.S. Cl. CPC A61K 47/6951; A61K 9/0048; A61K 47/10; A61K 47/183

(57) ABSTRACT

The present disclosure relates to ophthalmic compositions comprising solid complexes of active pharmaceutical ingredients, in particular kinase inhibitors and cyclodextrin, and to their uses in the treatment of posterior ocular conditions. More specifically, the disclosure relates to an aqueous composition comprising drug/cyclodextrin complexes of a tyrosine kinase inhibitor or a salt thereof, and a cyclodextrin, wherein said complexes have a complexation efficacy (CE) of more than 0.01, preferably more than 0.1 in the aqueous composition, and/or the tyrosine kinase inhibitor or a salt thereof has a ratio of the half maximal inhibitory concentration (IC₅₀) of the epidermal growth factor receptors (EGFR) to the half maximal inhibitory concentration (IC₅₀) of the vascular endothelial growth factor receptors (VEGFR2) that is greater than 2000, preferably greater than 5000.

### FIELD

The present disclosure relates to ophthalmic compositions containing solid complexes of active pharmaceutical ingredient and cyclodextrin, and to their uses in the treatment of posterior ocular conditions.

### BACKGROUND

Most ocular conditions can be treated and/or managed to reduce negative effects, including total blindness. To combat this significant problem, the World Health Organization (WHO) approved an action plan with the aim of reducing 25% of the world's avoidable visual impairments by 2019. In its efforts, the WHO plan to reduce the effects of ocular conditions such as diabetic retinopathy, glaucoma, and retinitis pigmentosa, which account for most cases of irreversible blindness worldwide. However, current treatments for ocular conditions are limited by the difficulty of delivering effective doses of drugs to target tissues in the eye.

Topical administration of eye drops is envisioned to be the preferred means of drug administration to the eye due to the convenience and safety of eye drops in comparison to other routes of ophthalmic drug administration such as intravitreal injections and implants (Le Bourlais, C., Acar, L., Zia, H., Sado, P. A., Needham, T., Leverge, R., 1998. Ophthalmic drug delivery systems—Recent advances. Progress in Retinal and Eye Research 17, 33-58). Drugs are mainly transported by passive diffusion from the eye surface into the eye and surrounding tissues where, according to Fick's law, the drug is driven into the eye by the gradient of dissolved drug molecules. The passive drug diffusion into the eye is hampered by three major obstacles (Gan, L., Wang, J., Jiang, M., Bartlett, H., Ouyang, D., Eperjesi, F., Liu, J., Gan, Y., 2013. Recent advances in topical ophthalmic drug delivery with lipid-based nanocarriers. Drug Discov. Today 18, 290-297; Loftsson, T., Sigurdsson, H. H., Konradsdottir, F., Gisladottir, S., Jansook, P., Stefansson, E., 2008. Topical drug delivery to the posterior segment of the eye: anatomical and physiological considerations. Pharmazie 63, 171-179; Urtti, A., 2006. Challenges and obstacles of ocular pharmacokinetics and drug delivery. Adv. Drug Del. Rev. 58, 1131-1135).

The first major obstacle is the aqueous drug solubility. In previously known ophthalmic compositions, only dissolved drug molecules can permeate through biological membranes into the eye. Accordingly, ophthalmic drugs must possess sufficient solubility in the aqueous tear fluid to permeate into the eye.

The second major obstacle is the rapid turnover rate of the tear fluid and the consequent decrease in concentration of dissolved drug molecules. Following instillation of an eye-drop (25-50 μl) onto the pre-corneal area, the greater part of the drug solution is rapidly drained from the eye surface and the tear volume returns to the normal resident volume of about 7 μl. Thereafter, the tear volume remains constant, but drug concentration decreases due to dilution by tear turnover and corneal and non-corneal drug absorption. The value of the first-order rate constant for the drainage of eye drops from the surface area is typically about 1.5 min-1 in humans after the initial rapid drainage. Normal tear turnover is about 1.2 μl/min in humans and the pre-corneal half-life of topically applied drugs is between 1 and 3 minutes (Sugrue, M. F., 1989. The pharmacology of antiglaucoma drugs. Pharmacology & Therapeutics 43, 91-138).

The third major obstacle is slow drug permeation through the membrane barrier, i.e. cornea and/or conjunctiva/sclera. The drug molecules must partition from the aqueous exterior into the membrane before they can passively permeate the membrane barrier. The result is that generally only few percentages of applied drug dose are delivered into the ocular tissues. The major part (50-100%) of the administered dose will be absorbed from the nasal cavity into the systemic drug circulation which can cause various side effects.

A fourth obstacle is that drug molecules that are administered to be delivered to the posterior segment of the eye and treat conditions of the posterior segment, may lead to serious side effects in the anterior segment of the eye.

The present disclosure seeks to assist with the WHO's plan for reducing avoidable visual impairments by providing an ophthalmic composition that overcomes the obstacles of passive drug diffusion into the eye and increases the bioavailability of a drug in the posterior segment of the eye, while reducing side effects in the anterior segment of the eye. It is one object of the present disclosure to provide a method for preparing an ophthalmic composition, which overcomes the major obstacles of passive drug diffusion by increasing the solubility of poorly soluble drugs. It is another object of the present disclosure to provide a method for preparing an ophthalmic composition which enhances the rate of migration of drug molecules from the aqueous exterior into the membrane to enable significantly more passive permeation of the membrane barrier towards the posterior segment of the eye. It is also an object of the present disclosure to provide methods of treating posterior ocular conditions while reducing side effects, in particular in the anterior segment of the eye.

### SUMMARY

Cyclodextrins are known to enhance the solubility and bioavailability of hydrophobic compounds. In aqueous solutions, cyclodextrins form inclusion complexes, non-inclusion complexes and aggregates of such complexes with many active pharmaceutical ingredients. Applicants have surprisingly found that the presence of salts and stabilizing agents in aqueous compositions comprising an active pharmaceutical ingredient allow for significantly higher concentration of active pharmaceutical ingredient in ophthalmic compositions.

Applicants also surprisingly found that certain ophthalmic compositions of the disclosure lead to a significantly higher delivery of active pharmaceutical ingredient to the posterior segment (i.e. retina and related tissues) of the eye.

The solutions of the disclosure attain a significant increase of the rate of migration of active pharmaceutical ingredients from the aqueous exterior into the membrane of the eye to enable significantly more passive permeation of the membrane barrier.

A higher concentration of active pharmaceutical ingredient in the ophthalmic compositions may carry the risk of stronger side effects, in particular in the anterior segment of the eye. Applicants surprisingly found out that tyrosine kinase inhibitors showing a certain half maximal inhibitory concentration (IC50) ratio of the vascular endothelial growth factor receptors (VEGFR2) to the epidermal growth factor receptors (EGFR) exhibit less side effects, while maintaining efficacy.

In a first aspect an aqueous composition is provided comprising drug/cyclodextrin complexes of a tyrosine kinase inhibitor or a salt thereof, and a cyclodextrin whereby said complexes have a complexation efficacy (CE) of more than 0.01 preferably more than 0.1 in the aqueous composition, and the half maximal inhibitory concentration (IC50) of said tyrosine kinase inhibitor or salt thereof for the vascular endothelial growth factor receptors (VEGFR2) is more than 2000 times greater, preferably more than 5000 times greater than that of the epidermal growth factor receptors (EGFR).

In a second aspect, said aqueous composition is provided for use in a topical treatment of retinal diseases.

In a third aspect, a method is provided for treating a condition of the posterior segment and/or the anterior segment of the eye in a subject in need thereof, said method comprising applying topically to the eye surface of said subject, said aqueous composition comprising as a tyrosine kinase inhibitor as the active principle, in an amount which delivers a therapeutically effective amount of said tyrosine kinase inhibitor to said segment or segments of the eye.

### BRIEF DESCRIPTION OF THE DRAWINGS

These and other features of this disclosure will now be described with reference to the drawings of certain embodiments which are intended to illustrate and not to limit the disclosure.

FIG. 1 depicts different types of phase-solubility diagrams, that is plots of total drug solubility vs total amount of cyclodextrin present in the complexation media (T. Higuchi, KA Connors: Phase-solubility techniques, Adv. Anal. Chem. Instrum. 4, 117-212, 1965).

FIG. 2 depicts corneal IC₅₀ graphs based on different peptides.

FIG. 3 depicts the dissolution profiles of acrizanib (AF1) and dovitinib (DF3) formulations in water.

FIG. 4 depicts the phase solubility profile of orantinib free acid in water at pH 2 to 11.

FIG. 5 depicts phase a stability study of axitinib.

FIG. 6 depicts the solubility of the axitinib free base in admixture with gamma-cyclodextrin (γCD) and various polymers, wherein HDMBR is hexadimethrine bromide (≥94% pure by titration with molecular weight 374 kDa) and the formulation vehicle consists of 0.1% (w/v) EDTA, 0.02% (w/v) benzalkonium chloride, and 0.05% (w/v) sodium chloride in pure water.

### DETAILED DESCRIPTION

Further aspects, features and advantages of the exemplary embodiments will become apparent from the detailed description which follows.

The patents, published applications and scientific literature referred to herein establish the knowledge of those with skill in the art and are hereby incorporated by reference in their entireties to the same extent as if each was specifically and individually indicated to be incorporated by reference.

As used herein, whether in a transitional phrase or in the body of a claim, the terms “comprise(s)” and “comprising” are to be interpreted as having an open-ended meaning. That is, the terms are to be interpreted synonymously with the phrases “having at least” or “including at least”. When used in the context of a method, the term “comprising” means that the method includes at least the recited steps, but may include additional steps. When used in the context of a composition, the term “comprising” means that the composition includes at least the recited features or components, but may also include additional features or components.

The terms “consists essentially of” or “consisting essentially of” have a partially closed meaning, that is, they do not permit inclusion of steps or features or components which would substantially change the essential characteristics of a method or composition; for example, steps or features or components which would significantly interfere with the desired properties of the compounds or compositions described herein, i.e., the method or composition is limited to the specified steps or materials and those which do not materially affect the basic and novel characteristics of the method or composition.

The terms “consists of” and “consists” are closed terminology and allow only for the inclusion of the recited steps or features or components.

As used herein, the singular forms “a,” “an” and “the” specifically also encompass the plural forms of the terms to which they refer, unless the content clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

The term “dissolved” or “substantially dissolved” is used herein to mean the solubilization of a solid in a solution. It can be considered that a solid is “dissolved” or “substantially dissolved” in a solution when the resulting solution is clear or substantially clear.

The term “clear” is used herein to mean a translucent or a subtranslucent solution. Thus, a “clear” solution has a turbidity measured according to ISO standards of ≤100 Nephelometric Turbidity Units (NTUs), preferably ≤50 NTUs.

The term “substantially clear” is used herein to mean a translucent or a subtranslucent solution. Thus, a “substantially clear” solution has a turbidity measured according to ISO standards of ≤100 Nephelometric Turbidity Units (NTUs).

As used herein, the term “cloudy” or “substantially cloudy” or refers to a solution having a turbidity measured according to ISO standards of greater than 100 NTUS.

As used herein, the term “milky” or “substantially milky” refers to a solution having a turbidity measured according to ISO standards of greater than 100 NTUs, preferably greater than 200 NTUs.

As used herein, the recitation of a numerical range for a variable is intended to convey that the variable can be equal to any values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

Technical and scientific terms used herein have the meaning commonly understood by one of skill in the art to which the present description pertains, unless otherwise defined. Reference is made herein to various methodologies and materials known to those of skill in the art. Standard reference works setting forth the general principles of pharmacology and pharmaceutics include Goodman and Gilman's The Pharmacological Basis of Therapeutics, 10^{th} Ed., McGraw Hill Companies Inc., New York (2001) and Remington, The Science and Practice of Pharmacy, 22^{nd} Ed., Philadelphia (2013).

As used herein the term “% by weight of a compound X based on the volume of the composition”, also abbreviated as “% w/v”, corresponds to the amount of compound X in grams that is introduced in 100 ml of the composition.

As used herein the term “microparticle” refers to a particle having a diameter D₅₀ of 1 μm or greater to about 500 μm. The term “nanoparticle” refers to a particle having a diameter D₅₀ of less than 1 μm.

In exemplary embodiments, the diameter, which can be D₅₀, is 1 μm or greater to about 500 μm; and the term “nanoparticle” refers to a particle having a D₅₀ of less than about 1 μm.

As used herein an “ocular condition” is a disease, ailment or other condition which affects or involves the eye, one of the parts or regions of the eye, or the surrounding tissues such as the lacrimal glands. Broadly speaking, the eye includes the eyeball and the tissues and fluids which constitute the eyeball, the periocular muscles (such as the oblique and rectus muscles), the portion of the optic nerve which is within or adjacent to the eyeball and surrounding tissues such as the lacrimal glands and the eye lids.

As used herein an “anterior ocular condition” is a disease, ailment or condition which affects or which involves an anterior (i.e. front of the eye) ocular region or site, such as a periocular muscle, an eye lid, lacrimal gland or an eyeball tissue or fluid which is located anterior to the posterior wall of the lens capsule or ciliary muscles.

Thus, an anterior ocular condition primarily affects or involves one or more of the following: the conjunctiva, the cornea, the anterior chamber, the iris, the lens, or the lens capsule, and blood vessels and nerves which vascularize or innervate an anterior ocular region or site. An anterior ocular condition is also considered herein as extending to the lacrimal apparatus. In particular, the lacrimal glands which secrete tears, and their excretory ducts which convey tear fluid to the surface of the eye. Furthermore, this includes neovascularization of the cornea, including corneal neovascularization associated with corneal inflammation, including herpes simplex keratitis, herpes zoster keratitis, bacterial corneal infections, fungal corneal infections and corneal graft rejection. It also includes iris neovascularization and neovascular glaucoma, which may be associated with retinal vein occlusion, diabetic retinopathy, other ischemic retinopathies and carotid stenosis.

Moreover, an anterior ocular condition affects or involves the posterior chamber, which is behind the retina but in front of the posterior wall of the lens capsule.

A “posterior ocular condition” is a disease, ailment or condition which primarily affects or involves a posterior ocular region or site such as the retina or choroid (in a position posterior to a plane through the posterior wall of the lens capsule), vitreous, vitreous chamber, retina, optic nerve (i.e. the optic disc), and blood vessels and nerves which vascularize or innervate a posterior ocular region or site.

Thus, a posterior ocular condition can include a disease, ailment or condition such as, for example, macular degeneration (such as non-exudative age-related macular degeneration and exudative age-related macular degeneration, also known as wet or neovascular age related macular degeneration); choroidal neovascularization; pachychoroidal disorders; polypoidal choroidal vasculopathy; acute macular neuroretinopathy; macular edema (such as cystoid macular edema and diabetic macular edema); Behcet's disease, retinal disorders, diabetic retinopathy (including proliferative diabetic retinopathy and diabetic macular edema; also non-proliferative diabetic retinopathy); retinal arterial occlusive disease; central retinal vein occlusion; branch retinal vein occlusion; sickle cell retinopathy; uveitic retinal disease also known as posterior uveitis, including macular edema associated with inflammation and neovascularization associated with inflammation; sarcoidosis retinal inflammation; sarcoidosis uveitis; syphilitic uveitis; systemic lupus erythematosus related inflammation in retina or retinal vessels; retinal detachment; proliferative vitreoretinopathy; ocular trauma which affects a posterior ocular site or location; a posterior ocular condition caused by or influenced by an ocular laser treatment; posterior ocular conditions caused by or influenced by a photodynamic therapy; photocoagulation; radiation retinopathy; epiretinal membrane disorders; branch retinal vein occlusion; anterior ischemic optic neuropathy.

The present description is concerned with and directed to ophthalmic compositions for topical drug delivery to the eye(s) and to methods for the treatment of a posterior ocular condition. In preferred embodiments, the ophthalmic compositions are used for the treatment of pathological states that arise or are exacerbated by ocular angiogenesis and vascular leakage, for example, in diabetic retinopathy (including background diabetic retinopathy, proliferative diabetic retinopathy and diabetic macular edema); age-related macular degeneration (AMD) (including neovascular (wet/exudative) AMD, dry AMD, and Geographic Atrophy); pathologic choroidal neo vascularization (CNV) from any mechanism (i.e. high myopia, trauma, sickle cell disease; ocular histoplasmosis, angioid streaks, traumatic choroidal rupture, drusen of the optic nerve, and some retinal dystrophies); pathologic retinal neovascularization from any mechanism (i.e., sickle cell retinopathy, retinopathy of prematurity, Eales disease, ocular ischemic syndrome, carotid cavernous fistula, familial exudative vitreoretinopa thy, hyperviscosity syndrome, idiopathic occlusive arteriolitis, birdshot retinochoroidopathy, retinal vasculitis, sarcoidosis, or toxoplasmosis); uveitis; retinal vein occlusion (central or branch); ocular trauma; surgery induced edema; surgery induced neovascularization; cystoid macular edema; ocular ischemia; retinopathy of prematurity; Coats disease; sickle cell retinopathy and/or neovascular glaucoma.

### Solid Complexes of Cyclodextrin and Active Pharmaceutical Ingredient

The composition comprises a solid complex comprising an active pharmaceutical ingredient and a cyclodextrin. The complex comprising an active pharmaceutical ingredient and a cyclodextrin may be referred to as an “active pharmaceutical ingredient/cyclodextrin complex” or a “drug/cyclodextrin complex”.

The solid complex of the composition may be a complex aggregate. The complex aggregate may correspond to an aggregate of a plurality of complexes, in particular a plurality of inclusion and non-inclusion complexes comprising an active pharmaceutical ingredient and a cyclodextrin.

According to one embodiment, the ophthalmic composition is a microsuspension. The term “microsuspension” is intended to mean a composition comprising solid complex microparticles suspended in a liquid phase.

In particular, the ophthalmic composition comprises a solid complex that has a diameter D₅₀ of about 0.1 μm to about 500 μm, in particular about 1 μm to about 100 μm, preferably 1 μm to about 50 μm. The diameter D₅₀ may be measured according to the test method described herein.

To form the present compositions with drug/cyclodextrin complexes or aggregates, the individual components are suspended in water, shortly heated and then kept under stirring at moderate temperatures for a given period. The compositions thus produced comprise a drug/cyclodextrin complex having an average D₅₀ particle size of about 0.1 μm to about 500 μm, in particular about 1 μm to about 100 μm, preferably 1 μm to about 50 μm. In certain embodiments, the compositions comprise about 70% to about 99% of the drug in microparticles and about 1% to about 30% of the drug in water-soluble nanoparticles, water-soluble drug/cyclodextrin complexes and dissolved free drug. The microparticles have an average D₅₀ particle size of less than 100 μm, preferably from about 1 μm to about 50 μm. In an exemplary embodiment, the composition is a microsuspension comprising about 80% of the drug in microparticles, and wherein said microparticles have an average diameter of about 1 μm to about 50 μm.

In one embodiment, the compositions comprise drug/cyclodextrin complex aggregates having a diameter of less than about 100 μm. In such embodiment, the compositions may comprise about 40% to about 99% of the drug in microparticles and about 1% to about 60% of the drug in dissolved nanoparticles, water-soluble drug/cyclodextrin complexes and dissolved free drug. The microparticles typically have an average diameter of about 1 μm to about 100 μm. In an exemplary embodiment, the microsuspension comprises about 80% of the drug to be in microparticles having an average diameter of about 1 μm to about 50 μm, and about 20% of the drug to be in water-soluble nanoparticles, water-soluble drug/cyclodextrin complexes and free drug.

In certain embodiments, the microsuspensions of the present disclosure may advantageously have about 10-fold to 1000-fold increase in dissolved active pharmaceutical agent concentration when compared to known microsuspensions.

Applicants have surprisingly found that such a high concentration of active pharmaceutical ingredient concentration may advantageously be achieved by the use of a drug in salt form, optionally in combination with chelating agents and surface active agents and, optionally with further additives as described below.

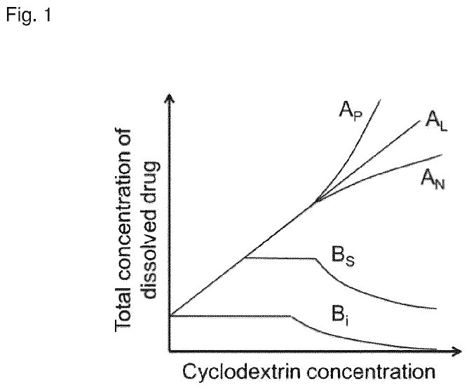

The two most important properties of the drug/cyclodextrin complexes are their stoichiometry and the numerical values of their stability constants. If m drug molecules (D) associate with n cyclodextrin molecules (CD) to form a complex (Dₘ/CDₙ) following overall equilibrium is attained:

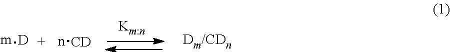

where Kₘᵢₙ is the stability constant of the drug/cyclodextrin complex. The stoichiometry of drug/cyclodextrin complexes and the numerical values of their stability constants are often obtained from phase-solubility diagrams where the drug solubility is monitored as a function of total cyclodextrin added to the complexation media (FIG. 1). If A_{L}-type (i.e., linear) phase-solubility diagram is obtained, then it can be assumed that the complex is first order with respect to cyclodextrin (n=1 in Eq. 1) and first or higher order with respect to the drug (m>=1). In this case the total concentration of dissolved drug (Sₜₒₜ) is equal to the sum of the apparent intrinsic drug solubility (S₀) and the concentration of drug in the dissolved complex (m·[Dₘ/CD]):

$${S}_{t o t} = {S}_{0} + m \cdot [ {D}_{m} / CD ] \qquad ( 2 )$$

The most common type of drug/cyclodextrin complexes in dilute aqueous solutions are 1:1 D/CD complexes. In this case, the slope of the linear phase-solubility diagram is less than unity and the following equation can be applied to calculate stability constant (K_{1:1}):

$${K}_{1 : 1} = \frac{Slope}{R \cdot ( 1 - Slope )} \qquad ( 3 )$$

Positive deviation from linearity (A_{P}-type phase-solubility diagram) suggests formation of higher order complex with respect to cyclodextrin. The stoichiometry of the system can be obtained by curve fitting with a quadratic model. A good fit to this model could suggest formation of a 1:2 drug/cyclodextrin complex:

$${S}_{t o t} = {S}_{0} + {K}_{1 : 1} \cdot {S}_{0} \cdot [ CD ] + {K}_{1 : 1} \cdot {K}_{1 : 2} \cdot {S}_{0} \cdot {[ CD ]}^{2} \qquad ( 4 )$$

where [CD] represents the concentration of free cyclodextrin. A third order model is suggestive of a 1:3 complex, etc. Here consecutive complexation is assumed where, for example, the 1:2 complex is formed when one additional cyclodextrin molecule forms a complex with an existing 1:1 complex. Phase-solubility studies are performed in aqueous solutions saturated with the drug where formation of higher-order complex aggregates is more likely than in diluted unsaturated solutions. AN-type profiles have been explained by changes in the complexation media and self-association of cyclodextrin molecules and/or their complexes at higher cyclodextrin concentrations. A-type phase-solubility diagrams are commonly observed in complexation media containing the water-soluble cyclodextrin derivatives such as 2-hydroxypropyl-α-cyclodextrin, 2-hydroxypropyl-β-cyclodextrin sulfobutyl ether β-cyclodextrin and 2-hydroxypropyl-γ-cyclodextrin. B-type phase-solubility diagrams (FIG. 1) suggest formation of poorly soluble complexes and they are commonly observed in aqueous complexation media containing the natural α-cyclodextrin,-cyclodextrin and γ-cyclodextrin. Bₛ-type phase-solubility diagrams are formed when the drug/cyclodextrin complex has limited solubility in the complexation medium with the profile plateau indicating the total drug solubility, i.e. the intrinsic drug solubility (S₀) plus the drug solubility in the form of cyclodextrin complexes. The ascending part of the profile can mathematically be treated as an A-type diagram and the previously described techniques used to gain information on the apparent stoichiometry of the complex. The decrease of total drug solubility at higher cyclodextrin concentrations which is manifested in the B-type profile is explained by completion of available drug in the complexation media. However, this decent is frequently observed when excess drug is available. Bᵢ-type profiles are similar to Bₛ-type except that the drug/cyclodextrin complexes formed are insoluble in the complexation media.

The intrinsic solubility (S₀) should be identical to the Y-intercept value of the phase-solubility diagram. However, this is rarely the case for poorly soluble drugs that tend to aggregate in aqueous solutions to form soluble dimers, trimers and higher order aggregates. Thus, complexation efficacy (CE) is frequently a better measure for comparison of solubilization effects of different cyclodextrins. If the slope of a linear phase-solubility diagram is less than unity, the CE can be calculated from the following equation (T. Loftsson, D. Hreinsdóttir and M. Masson: The complexation efficiency, J. Incl. Phenom. Macroc. Chem. 57, 545-552, 2007):

$$CE = {S}_{0} \cdot {K}_{1 : 1} = \frac{[ D / CD ]}{[ CD ]} = \frac{Slope}{1 - Slope} \qquad ( 5 )$$

where [D/CD] is the concentration of dissolved complex, [CD] is the concentration of dissolved free cyclodextrin and Slope is the slope of the linear phase-solubility profile. The complexation efficiency can be used to calculate the D:CD molar ratio, which can be correlated to the expected increase in formulation bulk:

$$Drug : γ - cyclodextrin molar ratio = 1 : \frac{( CE + 1 )}{CE} \qquad ( 6 )$$

The natural cyclodextrin and their derivatives, as well as their complexes, are all known to form aggregates, especially at elevated drug and cyclodextrin concentrations. Cyclodextrin-based solubilizing microparticles consist of guest/host complexes where the guest (e.g., drug) is poorly soluble in aqueous solutions (e.g., less than 1 mg/ml) and the aqueous solubility of the host (i.e. natural cyclodextrin) in the guest/host complex media is greater than 10-times the solubility of the guest but less than the solubility of the host. For example, the solubility of hydrocortisone in pure water at room temperature is about 0.1 mg/ml and that of γ-cyclodextrin under the same conditions is about 250 mg/ml. However, the solubility of hydrocortisone and γ-cyclodextrin in aqueous 3% (w/v) γ-cyclodextrin suspension saturated with hydrocortisone is 3 and 13 mg/ml, respectively (Phennapha Saokham, Thorsteinn Loftsson: γ-Cyclodextrin, International Journal of Pharmaceutics, 516, 278-292, 2017). Thus, the solubility of the guest (i.e. hydrocortisone) is increased by 30-fold while the host (i.e. γ-cyclodextrin) solubility is decreased by almost 20-fold.

It was thought that the ability of the technology to deliver drugs through biological membranes dependent on the ability of drug molecules to form cyclodextrin complexes, that is increasing with increasing K-value (Eq. 1). However, some drugs that have high K-values cannot be formulated in accordance to this technology and delivered through biomembranes such as from the surface of the eye into the eye. It has been discovered that the important parameter is the complexation efficacy (CE in Eq. 5). It can be difficult to obtain solid drug/cyclodextrin complexes if the CE is very low. Also, solid drug/cyclodextrin complexes of drugs with low CE are unstable in aqueous media where the drug is released from the complex to be precipitated as the pure drug. The optimum CE for successful development of kinase inhibitors according to this technology is greater than about 0.01, more preferable greater than about 0.05, and most preferable greater than about 0.1.

### Cyclodextrin

The composition comprises a cyclodextrin. The composition may comprise a mixture of cyclodextrins.

Cyclodextrins, which are also known as cycloamyloses, are produced from the enzymatic conversion of starch. They have a cyclic structure that is hydrophobic on the inside and hydrophilic on the outside. Because of the amphiphilic nature of the ring, cyclodextrins have been known to enhance the solubility, stability and bioavailability of hydrophobic compounds.

Cyclodextrins are cyclic oligosaccharides containing 6 (α-cyclodextrin), 7 (β-cyclodextrin), and 8 (γ-cyclodextrin) glucopyranose monomers linked via α-1,4-glycoside bonds. α-Cyclodextrin, β-cyclodextrin and γ-cyclodextrin are natural products formed by microbial degradation of starch. The outer surface of the doughnut shaped cyclodextrin molecules is hydrophilic, bearing numerous hydroxyl groups, but their central cavity is somewhat lipophilic (Kurkov, S. V., Loftsson, T., 2013. Cyclodextrins. Int J Pharm 453, 167-180; Loftsson, T., Brewster, M. E., 1996. Pharmaceutical applications of cyclodextrins. 1. Drug solubilization and stabilization. Journal of Pharmaceutical Sciences 85, 1017-1025). In addition to the three natural cyclodextrins numerous water-soluble cyclodextrin derivatives have been synthesized and tested as drug carriers, including cyclodextrin polymers (Stella, V. J., He, Q., 2008. Cyclodextrins. Tox. Pathol. 36, 30-42).

Cyclodextrins enhance the solubility and bioavailability of hydrophobic compounds. In aqueous solutions, cyclodextrins form inclusion complexes with many drugs by taking up a drug molecule, or more frequently some lipophilic moiety of the molecule, into the central cavity. This property has been used for drug formulation and drug delivery purposes. Formation of drug/cyclodextrin inclusion complexes, their effect on the physicochemical properties of drugs, their effect on the ability of drugs to permeate biomembranes and the usage of cyclodextrins in pharmaceutical products have been reviewed (Loftsson, T., Brewster, M. E., 2010. Pharmaceutical applications of cyclodextrins: basic science and product development. Journal of Pharmacy and Pharmacology 62, 1607-1621; Loftsson, T., Brewster, M. E., 2011. Pharmaceutical applications of cyclodextrins: effects on drug permeation through biological membranes.” J. Pharm. Pharmacol. 63, 1119-1135; Loftsson, T., Järvinen, T., 1999. Cyclodextrins in ophthalmic drug delivery. Advanced Drug Delivery Reviews 36, 59-79).

Cyclodextrins and drug/cyclodextrin complexes are able to self-assemble in aqueous solutions to form nano and micro-sized aggregates and micellar-like structures that are also able to solubilize poorly soluble active pharmaceutical ingredients through non-inclusion complexation and micellar-like solubilization (Messner, M., Kurkov, S. V., Jansook, P., Loftsson, T., 2010. Self-assembled cyclodextrin aggregates and nanoparticles. Int. J. Pharm. 387, 199-208). In general, hydrophilic cyclodextrin derivatives, such as 2-hydroxypropyl-β-cyclodextrin and 2-hydroxypropyl-γ-cyclodextrin, and their complexes are freely soluble in water. On the other hand, the natural α-cyclodextrin, β-cyclodextrin and γ-cyclodextrin have limited solubility in pure water or 129.5±0.7, 18.4±0.2 and 249.2±0.2 mg/ml, respectively, at 25° C. (Sabadini E., Cosgrovea T. and do Carmo Egídio F., 2006. Solubility of cyclomaltooligosaccharides (cyclodextrins) in H₂O and D₂O: a comparative study. Carbohydr Res 341, 270-274). Solubilities of their complexes can be higher or lower than that of the pure cyclodextrins. It is known that their solubility increases somewhat with increasing temperature (Jozwiakowski, M. J., Connors, K. A., 1985. Aqueous solubility behavior of three cyclodextrins. Carbohydr. Res., 143, 51-59). Due to the limited solubility of their complexes, the natural cyclodextrins most often display Bₛ-type or Bᵢ-type phase-solubility diagrams (Brewster M. E., Loftsson T., 2007, Cyclodextrins as pharmaceutical solubilizers. Adv. Drug Deliv. Rev., 59, 645-666).

In a preferred embodiment, the cyclodextrin is α-cyclodextrin, β-cyclodextrin, γ-cyclodextrin, or combinations thereof.

In a particularly preferred embodiment, the cyclodextrin is γ-cyclodextrin. γ-Cyclodextrin has a higher solubility in water compared to that of α-cyclodextrin and β-cyclodextrin. Moreover, γ-cyclodextrin is prone to hydrolysis into glucose and maltose subunits by α-amylase in the tear fluid and the gastrointestinal tract.

The amount of cyclodextrin in the ophthalmic composition of the disclosure, typically γ-cyclodextrin may be from 0.25% (w/v) to 40% (w/v) in particular 10% (w/v) to 30% (w/v), more particularly 15% (w/v) to 25% (w/v) weight cyclodextrin based on the volume of the composition.

### Active Pharmaceutical Ingredient

The present compositions comprise an active pharmaceutical ingredient.

The active pharmaceutical ingredient may be referred to as a “drug”. In the context of the disclosure, the active pharmaceutical ingredient is an ophthalmic drug, i.e. a compound that exhibits a therapeutic effect when administered in a sufficient amount to a patient suffering from an ocular condition.

In certain embodiments, the composition may comprise an active pharmaceutical ingredient selected from the group consisting of a kinase inhibitor such as afatinib, alectinib, anlotinib, axitinib, BMS-794833 (N-(4-((2-amino-3-chloropyridin-4-yl)oxy)-3-fluorophenyl)-5-(4-fluorophenyl)-4-oxo-1,4-dihydropyridine-3-carboxamide), binimetinib, bosutinib, brigatinib, cabozantinib, cediranib, cobimetinib, crizotinib, dasatinib, dovitinib, entrectinib, erlotinib, everolimus, gefitinib, ibrutinib, imatinib, lapatinib, lenvatinib, lestaurtinib, linifanib, masitinib, momelotinib, motesanib, neratinib, nilotinib, nintedanib, olmutinib, orantinib, osimertinib, pacritinib, PD173074 (N-[2-[4-(Diethylamino)butyl]amino]-6-(3,5-dimethoxyphenyl)pyrido[2,3-d]pyrimidin-7-yl]-N′-(1,1-dimethylethyl) urea), pazopanib, ponatinib, regorafenib, rociletinib, ruxolitinib, semaxinib, selumetinib, sorafenib, sunitinib, temsirolimus, tivozanib, toceranib, tofacitinib, trametinib, vandetanib, vemurafenib, and ZM323881 (5-((7-Benzyloxyquinazolin-4-yl)amino)-4-fluoro-2-methylphenol).

The active pharmaceutical ingredient for use in the nano- and microparticles in the exemplary embodiments can be selected from, but are not limited to, the group consisting of a kinase inhibitor such as afatinib, alectinib, anlotinib, axitinib, BMS-794833 (N-(4-((2-amino-3-chloropyridin-4-yl)oxy)-3-fluorophenyl)-5-(4-fluorophenyl)-4-oxo-1,4-dihydropyridine-3-carboxamide), binimetinib, bosutinib, brigatinib, cabozantinib, cediranib, cobimetinib, crizotinib, dasatinib, dovitinib, entrectinib, erlotinib, everolimus, gefitinib, ibrutinib, imatinib, lapatinib, lenvatinib, lestaurtinib, linifanib, masitinib, momelotinib, motesanib, neratinib, nilotinib, nintedanib, olmutinib, orantinib, osimertinib, pacritinib, PD173074 (N-[2-[[4-(Diethylamino)butyl]amino]-6-(3,5-dimethoxyphenyl)pyrido [2,3-d]pyrimidin-7-yl]-N′-(1,1-dimethylethyl) urea), pazopanib, ponatinib, regorafenib, rociletinib, ruxolitinib, semaxinib, selumetinib, sorafenib, sunitinib, temsirolimus, tivozanib, toceranib, tofacitinib, trametinib, vandetanib, vemurafenib, and ZM323881 (5-((7-Benzyloxyquinazolin-4-yl)amino)-4-fluoro-2-methylphenol).

Protein kinase inhibitors, such as tyrosine kinase inhibitors, are enzyme inhibitors that block the action of one or more protein kinases that are able to add a phosphate group to a protein and, thus, alter its function. Kinase inhibitors (KI) are frequently used as anticancer drugs or anti-inflammatory drugs. In ophthalmology kinase inhibitors can be used to treat disorders associated with microvascular pathology, increased vascular permeability and intraocular neovascularization, including age-related macular degeneration (AMD), diabetic retinopathy (DR) and diabetic macular edema (DME). The three main types of growth factor receptors of the tyrosine kinases are the epidermal growth factor receptors (EGFR), the platelet-derived growth factor receptors and the vascular endothelial growth factor receptors (VEGFR). VEGFR family members include VEGFR1, VEGFR2 and VEGFR3. Among them, VEGFR2 is the most important in mediating the biological effect of vascular endothelial growth factor and inhibitors of VEGFR2 are the most relevant for a treatment of AMD, DR and DME. Hence, as used herein, the term “tyrosine kinase inhibitors” refers to compound inhibitors of at least VEGFR receptors.

On the other hand, inhibition of EGFR results in various ocular side effects such as corneal thinning and erosion. Ocular side effects are mainly associated with the eye surface and the anterior section (i.e. the kinase inhibitor concentration in the anterior section) of the eye while the therapeutic effect is associated with the kinase concentration in the retina (or the posterior section of the eye).

Typically, in an embodiment of the present ophthalmic solutions tyrosine kinase inhibitors may have a 2000-fold or higher affinity for VEGFR2 than for EGFR to be administered topically to the eye in the form of aqueous eye drops. In a preferred embodiment, tyrosine kinase inhibitors may have a 5000-fold or higher affinity for VEGFR2 than for EGFR to be administered topically to the eye in the form of aqueous eye drops.

A table of relevant VEGFR inhibitors, their IC₅₀ (nM) values for VEGFR2 and EGFR and the EGFR/VEGFR2 IC₅₀ (nM) ratio obtained from Selleckchem (https://www.selleckchem.com/), Tocris Bioscience (https://www.tocris.com/) and TargetMol (https://www.targetmol.com/) is given below:

| | IC₅₀ | | EGFR/ |
|---|---|---|---|
| Kinase | VEGFR2 | EGFR | VEGFR2 |
| inhibitor | (nM) | (nM) | ratio |
| Acrizanib | 19 | 1000 | 53 |
| Altiratinib | 9.2 | 3300 | 360 |
| Anlotinib | 0.2 | 2000 | 10,000 |
| Axitinib | 0.2 | 1000 | 5000 |
| BFH772 | 3 | 10,000 | 3333 |
| Brivanib | 25 | 10,000 | 400 |
| Cabozantinib | 0.035 | 1000 | 29,000 |
| Cediranib | 1 | 1600 | 1600 |
| Dovitinib | 13 | 2200 | 169 |
| Foretinib | 0.86 | 3000 | 3500 |
| Fruquintinib | 35 | 30,000 | 857 |
| Ki8751 | 4 | 10,000 | 2500 |
| Lenvatinib | 4 | 6500 | 1625 |
| Linifanib | 1.4 | 50,000 | 36,000 |
| Motesanib | 3 | 3000 | 1000 |
| Nintedanib | 1.4 | 50000 | 36,000 |
| Orantinib | 2.1 | 100,000 | 50,000 |
| OSI-930 | 9 | 10,000 | 1000 |
| Ponatinib | 1.5 | 1000 | 700 |
| Regorafenib | 4.2 | 1000 | 250 |
| Semaxanib | 160 | 100,000 | 600 |
| Sitravatinib | 5 | 5000 | 1000 |
| Sorafenib | 90 | 10,000 | 100 |
| Tivozanib | 6.5 | 1000 | 150 |
| Vandetanib | 40 | 500 | 13 |
| ZM 323881 | 2 | 50,000 | 25,000 |

Of the 26 kinase inhibitors reviewed, ten had EGFR/VEGFR2 IC₅₀ ratio greater than 2000 and seven greater or equal to 5000.

According to a preferred embodiment the composition may comprise a tyrosine kinase inhibitor which has a ratio of the half maximal inhibitory concentration (IC₅₀) of the epidermal growth factor receptors (EGFR) to the half maximal inhibitory concentration (IC₅₀) of the vascular endothelial growth factor receptors (VEGFR2) that is greater than 2000, preferably greater than 5000. In certain preferred embodiments, the composition may comprise a salt form of said tyrosine kinase inhibitor.

According to a preferred embodiment the composition may comprise a tyrosine kinase inhibitor having a pKa of 2 to 8.

Preferred tyrosine kinase inhibitors for use in the composition of the present disclosure are nintedanib, cabozantinib, axitinib, anlotinib, linifanib, and orantinib. Most preferred tyrosine kinase inhibitors are nintedanib, orantinib and axitinib. The formulas are given below:

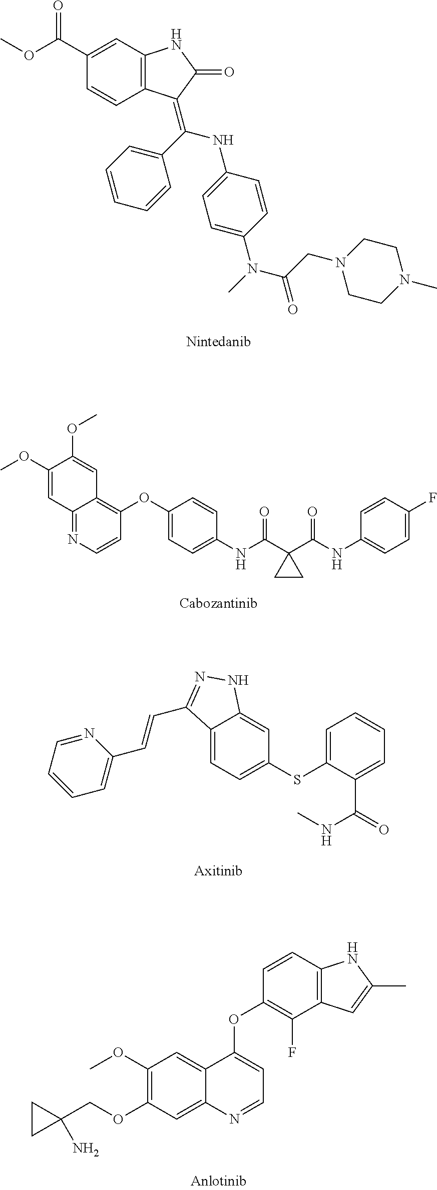

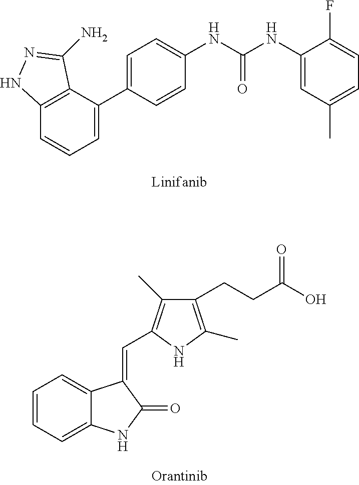 [SMILES: Cc1ccc(F)c(NC(=O)Nc2ccc(-c3cccc4[nH]nc(N)c34)cc2)c1 ; Cc1[nH]c(/C=C2\C(=O)Nc3ccccc32)c(C)c1CCC(=O)O]

The compositions may comprise the active pharmaceutical ingredient in salt form, i.e. as its inorganic or organic salt selected from the group consisting of propionate, acetate, 2,5-dihydroxybenzoate, citrate, malonate, sulfate, bisulfate, benzoate, maleate, tosylate, fumarate, succinate, tartrate, lactate, glycolate, phosphate, pyrophosphate, benzenesulfonate, ascorbate, chloride, bromate, malate, propionate, oxalate, isobutyrate, benzoate, sulfonate, mesylate, esylate and pyroglutamate, as well as their isomers.

Preferably the salt is selected from the group consisting of acetate, lactate, chloride, malate, esylate, maleate, aspartate, sodium, potassium.

The composition may comprise nintedanib as the free base or an esylate salt (i.e. ethanesulfonate salt) or chloride salt or a bromide salt, preferably as an esylate salt.

The composition may comprise cabozantinib as the free base or a malate salt or chloride salt, preferably as a malate salt.

The composition may comprise axitinib as the free base or an esylate or a tosylate salt, preferably as an esylate salt.

The composition may comprise orantinib as the free acid or a sodium salt or a potassium salt.

The concentration of active pharmaceutical ingredient in the final (ready-to-use) compositions may be from about 0.1 mg/ml to about 100 mg/mL, in particular from about 1 mg/mL to about 50 mg/mL, more particular from about 5 mg/ml to about 30 mg/ml as a free base or in salt form.

In exemplary embodiments, the active pharmaceutical ingredient is present in the final compositions at a concentration of about 1 mg/mL to about 50 mg/mL as a free base or in salt form.

The compositions may have about 10-fold to about 1000-fold increase in dissolved active pharmaceutical ingredient concentration when compared to compositions prepared according to known methods.

When the active pharmaceutical ingredient is dissolved in salt form, the concentration in the final composition may be increased to 0.5 to 5% (w/v), preferably 1 to 4% (w/v), more preferably 1.0 to 3.0% (w/v), when compared to the dissolution of the free base in the final composition. In particular, when the active pharmaceutical ingredient is dissolved in salt form in combination with one or more of a chelating agent, a surface active agent and optionally other excipients, it is present in the final composition at a concentration of 0.5 to 5% (w/v), preferably 1 to 4% (w/v), more preferably 1.0 to 3.0% (w/v) (weight of drug and volume of solution).

In particular 40 to 98% by weight, preferably 50 to 95% by weight, more preferably 60% to 90% by weight of the active pharmaceutical ingredient in the composition may be in the form of a solid complex of active pharmaceutical ingredient and cyclodextrin. The solid complex may comprise a salt of the active pharmaceutical ingredient and a chelating agent.

In particular, 2 to 60% by weight, more preferably 5 to 50% by weight, most preferably 10 to 40% by weight, of the active pharmaceutical ingredient in the composition may be in dissolved form. The dissolved form includes uncomplexed active pharmaceutical ingredient that is dissolved in the liquid phase and complexes of active pharmaceutical ingredient and cyclodextrin that are dissolved in the liquid phase as well as water-soluble nanoparticles consisting of drug/cyclodextrin complex aggregates. The dissolved forms may include chelating agents.

Preferably less than 5%, preferable less than 2% and more preferably less than 0.5% by weight of the active pharmaceutical ingredient in the composition may be in uncomplexed solid form. As such, the composition may be substantially free of solid uncomplexed particles of active pharmaceutical ingredient.

In one embodiment, the compositions are microsuspensions and may comprise about 70% to about 99% of the active pharmaceutical ingredient in microparticles and about 1% to about 30% of the active pharmaceutical ingredient in nanoparticles. More particularly, the microsuspension may comprise about 80% of the active pharmaceutical ingredient in microparticles having an average D₅₀ of the particles in the solid phase of from about 0.1 μm to about 500 μm, in particular 1 μm to 100 μm, more preferably 1 μm to 50 μm and about 20% of the active pharmaceutical ingredient in nanoparticles.

In another embodiment, the microsuspension may comprise about 40% to about 99% of the active pharmaceutical ingredient in microparticles and about 1% to about 60% of the active pharmaceutical ingredient in water-soluble nanoparticles or water-soluble active pharmaceutical ingredient/cyclodextrin complexes. In particular, the microsuspension may comprise about 80% to about 90% of the active pharmaceutical ingredient in microparticles having an average D₅₀ of the particles in the solid phase of about 1 μm to about 100 μm, and about 10% to about 20% of the active pharmaceutical ingredient in nanoparticles or water-soluble active pharmaceutical ingredient/cyclodextrin complexes.

### Polymer

The compositions may further comprise a polymer.

In particular, said polymer may be a water-soluble polymer. Moreover, said polymer may be a surface active polymer. The term “surface active polymer” is intended to mean a polymer that exhibits surfactant properties. The polymer enhances the physical stability of the composition. As such, the composition is less prone to sedimentation of the solid complex when it comprises a polymer. The polymer may thus be considered as a polymeric stabilizing agent. Surface active polymers may, for example, comprise hydrophobic chains grafted to a hydrophilic backbone polymer; hydrophilic chains grafted to a hydrophobic backbone; or alternating hydrophilic and hydrophobic segments. The first two types are called graft copolymers and the third type is named block copolymer.

In one embodiment, the composition comprises a polymer selected from the group consisting of a polyoxyethylene fatty acid ester; a polyoxyethylene alkylphenyl ether; a polyoxyethylene alkyl ether; a cellulose derivative such as alkyl cellulose, hydroxyalkyl cellulose and hydroxyalkyl alkylcellulose; a carboxyvinyl polymer such as a carbomer, for example Carbopol 971 and Carbopol 974; a polyvinyl polymer; a polyvinyl alcohol; a polyvinylpyrrolidone; a copolymer of polyoxypropylene and polyoxyethylene; tyloxapol; and combinations thereof.

Examples of suitable polymers include, but are not limited to, polyethylene glycol monostearate, polyethylene glycol distearate, hydroxypropyl methylcellulose, hydroxypropyl cellulose, polyvinylpyrrolidone, polyoxyethylene lauryl ether, polyoxyethylene octyldodecyl ether, polyoxyethylene stearyl ether, polyoxyethylene myristyl ether, polyoxyethylene oleyl ether, sorbitan esters, polyoxyethylene hexadecyl ether (e.g., cetomacrogol 1000), polyoxyethylene castor oil derivatives, polyoxyethylene sorbitan fatty acid esters (e.g., Tween 20 and Tween 80 (ICI Specialty Chemicals)); polyethylene glycols (e.g., Carbowax 3550 and 934 (Union Carbide)), polyoxyethylene stearates, carboxymethylcellulose calcium, carboxymethylcellulose sodium, methylcellulose, hydroxyethylcellulose, hydroxypropyl methylcellulose, cellulose, polyvinyl alcohol (PVA), poloxamers (e.g., Pluronics F68 and FI08, which are block copolymers of ethylene oxide and propylene oxide); poloxamines (e.g., Tetronic 908, also known as Poloxamine 908, which is a tetrafunctional block copolymer derived from sequential addition of propylene oxide and ethylene oxide to ethylenediamine (BASF Wyandotte Corporation, Parsippany, N.J.)); Tetronic 1508 (T-1508) (BASF Wyandotte Corporation), Tritons X-200, which is an alkyl aryl polyether sulfonate (Rohm and Haas); PEG-derivatized phospholipid, PEG-derivatized cholesterol, PEG-derivatized cholesterol derivative, PEG-derivatized vitamin A, PEG-derivatized vitamin E, random copolymers of vinyl pyrrolidone and vinyl acetate, combinations thereof and HDMBR (hexadimethrine bromide).

Preferred examples of polymers are tyloxapol, a copolymer of polyoxypropylene and polyoxyethylene, polyalkylenglycol, hydroxyalkylcellulose, hydroxyalkyl alkylcelllulose, and polyvinylalcohol.

Tyloxapol is a 4-(1,1,3,3-tetramethylbutyl) phenol polymer with formaldehyde and oxirane.

More particularly, the copolymer of polyoxypropylene and polyoxyethylene may be a triblock copolymer comprising a hydrophilic block (polyoxyethylene)-hydrophobic block (polyoxypropylene)-hydrophilic block (polyoxyethylene) configuration, also named poloxamer.

In one embodiment, the composition of the disclosure comprises a polymer which is a poloxamer. Poloxamers can include any type of poloxamer known in the art. Poloxamers include poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate and poloxamer 182 dibenzoate.

Especially useful polymers as stabilizers are poloxamers. Poloxamers can include any type of poloxamer known in the art. Poloxamers include poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate and poloxamer 182 dibenzoate.

### Chelating Agent

The compositions may further comprise chelating agents. Chelating agents contribute to the stability of the dissolved and suspended solid cyclodextrin/active pharmaceutical agent complexes. The chelating agents stabilize the compositions. They may solubilize counter ions. They may stabilize the pH to a limited degree.

Examples of chelating agents are divalent and polyvalent carboxylic acids and their salts. Preferred examples are ethylenediaminetetraacetic acid (EDTA), 2,2′,2″-nitrilotriacetic acid (NTA), iminodisuccinic acid (IDS), polyaspartic acid, S,S-ethylenediamine-N,N′-disuccinic acid (EDDS), methylglycinediacetic acid (MGDA), L-Glutamic acid N,N-diacetic acid, fumaric acid, tartaric acid, oxalic acid, maleic acid, malic acid, succinic acid and citric acid. EDTA is particularly preferred as a stabilizer, because it also contributes to pH stability. In an exemplary embodiment, the EDTA can be ethylenediaminetetraacetic acid disodium salt.

The amount of the chelating agent in the composition may be 0.1% (w/v) to 5% (w/v), in particular 0.3% (w/v) to 3% (w/v), more particularly 0.5% (w/v) to 2% (w/v) by weight of chelating agent based on the volume of the composition.

### Opthalmically Acceptable Medium

The compositions comprise an ophthalmically acceptable medium. The term “ophthalmically acceptable medium” is intended to mean a medium suitable for ophthalmic, topical administration of the composition, so as to be compatible with the eye and tear fluid. The ophthalmically acceptable medium is preferably a liquid. The ophthalmically acceptable medium may notably comprise purified water in at least 60% (w/v). In particular, the ophthalmically acceptable medium does not comprise any other solvent than water.

The compositions will typically have a pH in the range 3.5 to 9, preferably 4.5 to 7.5. The compositions will typically have osmolality of 200 to 450 milliosmoles per kilogram (mOsm/kg), more preferably 240 to 360 mOsm/kg.

According to a preferred embodiment the ophthalmically acceptable medium comprises water and optionally an additive selected from the group consisting of a preservative, a stabilizing agent, an electrolyte, a buffering agent, and combinations thereof.

In particular, the ophthalmically acceptable medium may comprise a preservative. A preservative may be used to limit bacterial proliferation in the composition.

Suitable examples of preservative are sodium bisulfite, sodium bisulfate, sodium thiosulfate, benzalkonium chloride, chlorobutanol, thimerosal, phenylmercuric acetate, phenylmercuric nitrate, methylparaben, phenylethyl alcohol, sorbic acid and its salts, and combinations thereof. Preferably, the preservative is benzalkonium chloride.

The amount of preservative in the composition of the disclosure may be 0 to 1%, in particular 0.001 to 0.5%, more particularly 0.005 to 0.1%, even more particularly 0.01 to 0.04%, by weight of preservative based on the volume of the composition. In certain embodiments, the composition does not contain any preservative.

In certain embodiments, the ophthalmically acceptable medium may comprise tonicity adjusting agent that is used to make the composition isotonic.

Examples of suitable tonicity adjusting agents include sodium chloride, potassium chloride, mannitol, dextrose, glycerin, and combinations thereof. Preferably, the electrolyte is sodium chloride.

The amount of tonicity adjusting agent in the composition of the disclosure may be 0.01 to 5% by weight of tonicity adjusting agent based on the volume of the composition. The concentration range may depend on the type of tonicity adjusting agent. For electrolytes like sodium chloride and potassium chloride the concentration range might be from 0.01% to 0.9% (w/v), while for non-electrolytes like mannitol and dextrose the range might be 0.1% to 5% (w/v).

### Method of Production

The present compositions may be prepared by suspending the individual components in water followed by heating in a closed container for about 20 min at 121° C. to form an essentially clear solution. Then the solution is allowed to cool to ambient temperature followed by equilibration at 22-23° C. under constant agitation. During the equilibration the pH of the compositions is adjusted to about 4.5 to about 7.5 with aqueous 0.1 N hydrochloric acid (HCl) solution and aqueous 1.0 N sodium hydroxide (NaOH) solution and the volume adjusted with distilled water.

### Uses of the Composition

The ophthalmic compositions of the disclosure may be for use in the treatment of an ocular condition, in particular a posterior ocular condition, more particularly for the treatment of pathological states that arise or are exacerbated by ocular angiogenesis and vascular leakage, for example, in diabetic retinopathy (including background diabetic retinopathy, proliferative diabetic retinopathy and diabetic macular edema); age-related macular degeneration (AMD) (including neovascular (wet/exudative) AMD, dry AMD, and Geographic Atrophy); pathologic choroidal neo vascularization (CNV) from any mechanism (i.e. high myopia, trauma, sickle cell disease; ocular histoplasmosis, angioid streaks, traumatic choroidal rupture, drusen of the optic nerve, and some retinal dystrophies); pathologic retinal neovascularization from any mechanism (i.e., sickle cell retinopathy, retinopathy of prematurity, Eales disease, ocular ischemic syndrome, carotid cavernous fistula, familial exudative vitreoretinopa thy, hyperviscosity syndrome, idiopathic occlusive arteriolitis, birdshot retinochoroidopathy, retinal vasculitis, sarcoidosis, or toxoplasmosis); uveitis; retinal vein occlusion (central or branch); ocular trauma; surgery induced edema; surgery induced neovascularization; cystoid macular edema; ocular ischemia; retinopathy of prematurity; Coats disease; sickle cell retinopathy and/or neovascular glaucoma.

The ophthalmic composition may in particular be used for the treatment of macular edema. In this case, the ophthalmic composition may be topically administered to the eye in an amount of 1 drop of composition three times per day. The amount of kinase inhibitor in said composition may be from of 0.5 to 5% (w/v), preferably 1 to 4% (w/v), more preferably 1.0 to 3.0% (w/v) weight of kinase inhibitor based on the volume of the composition.

The compositions of the disclosure do not need to be administered as frequently as known topical compositions. Indeed, due to the higher concentration of the active pharmaceutical ingredient in the composition and longer duration of delivery, the bioavailability of the active pharmaceutical ingredient in the posterior segment is significantly increased, so that a lower frequency of administration is possible, increasing patient compliance.

The present disclosure also covers the use of the compositions as an eye drop solution, so that depending on the indication and its severity, respectively, the solutions may be administered instead of or in addition to ophthalmic injection solutions, thereby significantly enhancing patient compliance and clinical outcome.

### Measuring Methods

### Diameter

The diameter of a particle, such as a solid complex of active pharmaceutical ingredient and cyclodextrin, can correspond to the D₅₀ diameter of the particle. Diameter D₅₀ is also known as the median diameter or the medium value of the particle size distribution. Diameter D₅₀ corresponds to the value of the particle diameter at 50% in the cumulative distribution. For example, if D₅₀ is 5 μm, then 50% of the particles in the sample are larger than 5 μm, and 50% smaller than 5 μm. Diameter D₅₀ is usually used to represent the particle size of a group of particles.

The diameter and/or size of a particle or complex can be measured according to any method known to those of ordinary skill in the art. For example, the diameter D₅₀ is measured by laser diffraction particle size analysis. Generally, there are a limited number of techniques for measuring/evaluating cyclodextrin/drug particle or complex diameter and/or size. In particular, persons of ordinary skill in this field know that the physical properties (e.g. particle size, diameter, average diameter, mean particle size, etc.) are typically evaluated/measured using such limited, typical known techniques. For example, such known techniques are described in Int. J. Pharm. 493 (2015), 86-95. In addition, such limited, known measurement/evaluation techniques were known in the art as evidenced by other technical references such as, for example, European Pharmacopoeia (2.9.31 Particle size analysis by laser diffraction, January 2010), and Saurabh Bhatia, Nanoparticles types, classification, characterization, fabrication methods and drug delivery applications, Chapter 2, Natural Polymer Drug Delivery Systems, PP. 33-94, Springer, 2016, which are also incorporated by reference herein in their entireties.

For particle size of complexes comprising an active pharmaceutical ingredient, the particle size is measured by laser diffraction particle size analysis according to Pharm. Eur. 2.9.31 applying the following parameters:

MasterSizer method description.

| | |
|---|---|
| Instrument | Malvern MasterSizer ® 3000 Hydro MV |
| Software | Mastersizer v 3.70 |
| Particle type | Opaque particle (Fraunhofer |
| | approximation) |
| Dispersant | Type I water |
| Refractive index of the | 1.33 |
| dispersant | |
| Background | 10 sec |
| measurements duration | |
| Sample measurements | 1 sec |
| duration | |
| Number of measurements | 15 |
| Obscuration lower limit | 2% |
| Obscuration higher limit | 20% |
| Measurement mode | Auto-start with stabilization time of |
| | 0.2 sec |
| Stirrer speed | 1200 rpm |
| Analysis model | General purpose |
| Sensitivity | Normal, with “fine powder” mode on |
| Result type | Volume distribution |
| Sample preparation | Manual homogenization by shaking |
| Sample size | 500 μl |
| Cleaning between | Triple rinsing with tap water and |
| measurements | once with type II water |

Analysis of samples with Olympus BX43 light microscope was done in accordance with Pharm. Eur. 2.9.37. 1 μl of manually homogenized eye drops was scanned under different magnification (up to 40×). Treatment of microscopic photos was done by means of equipped Olympus LC30 digital camera and LCmicro® v2.2 software.

### Percentage of Drug in Solid Complex and Percentage of Dissolved Drug

The amount of drug in the form of solid complexes and the amount of dissolved drug is obtained by centrifuging the composition at 6000 rpm at a temperature of 22-230 C for 20-30 minutes.

The amount of dissolved drug corresponds to the amount of drug in the supernatant as measured by high-performance liquid chromatography.

The percentage of drug in the form of a solid complex is obtained with the following formula:

$$\% drug □ solid complex = \frac{( total drug - dissolved drug )}{total drug} \times 100$$

wherein
“total drug” is the total amount of drug introduced in the composition in mg/ml; and
“dissolved drug” is the amount of drug in the supernatant in mg/mL.
The percentage of dissolved drug is obtained with the following formula:

$$\% dissolved drug = 100 - \% drug □ solid complex$$

### Determination of IC₅₀ (nM) Values for VEGFR2 and EGFR of Kinase Inhibitors

### Materials:

Assays were performed with tyrosine kinase peptide microarrays (PTK PamChips®) catalogue #86401 and reagents commercially available from PamGene International BV ('s-Hertogenbosch, the Netherlands). The PamChip® peptide arrays measure the ability of active recombinant kinases to phosphorylate specific peptides imprinted on multiplex peptide arrays (ref: PMID: 19344656). The PamChip contains 194 covalently coupled peptides derived from known human phosphorylation sites.

The inhibitors nintedanib, cabozantinib malate and axitinib were from SelleckChem (Houston, TX, USA).

EGFR (C-terminal fragment, amino acids H672-A1210) and VEGFR2 (C-terminal fragment, amino acids D807-V1356) were provided by Proqinase (Freiburg Germany). Mammalian Protein Extraction Reagent (M-PER) (Cat no. #78501), Halt Phosphatase Inhibitor Cocktail (Cat no. #78420) and Halt Protease Inhibitor Cocktail EDTA free (Cat no. #87785) were ordered from ThermoFisher Scientific (MA, USA).

### Methods:

Inhibitors were dissolved in DMSO and diluted in DMSO to 50× the final concentration. Recombinant kinases were diluted in Mammalian Protein extraction buffer (M-PER). The standard assay mix was supplemented with protease and phosphatase inhibitor cocktail (1/100 diluted) and MgCl2 was added to a final concentration of 17.5 mM. The optimal sample input was determined by testing a concentration range of kinase.

### PamChip® Assay:

Measurements of kinome activity were performed on a PamStation®-12 by PamGene (PMID: 30610604). Briefly, the arrays on the PamChip® were incubated with 2% BSA blocking buffer for 30 cycles (15 min) to prevent non-specific binding, followed by three times washing with protein kinase assay buffer (proprietary information). Subsequently, the PamChip protein tyrosine kinase (PTK) array was processed in a single-step reaction in which about 0.5 ug of recombinant kinases was dispensed onto PTK array dissolved in protein kinase buffer (proprietary information) and additives including 25 μM ATP and 0.01% BSA, supplemented with 4 μl protein kinase (PK)-additive (PamGene International BV), 10 mM Dithiothreitol (DTT, Fluka, Sigma-Aldrich, St. Louis, MO, USA) and fluorescein isothiocyanate (FITC) labeled anti-phosphotyrosine antibody (PamGene International BV, 's-Hertogenbosch, The Netherlands) in a final volume of 40 μL (assay master mix).

DMSO or kinase inhibitors were added to the assay mix to yield 2% final DMSO concentration. The inhibitor concentration varied from 1 nM to 10 UM for VEGFR2, for EGFR 10 UM and 200 UM inhibitor was tested.

Peptide phosphorylation was monitored during the incubation with assay mixture, by taking images every 2.5 minutes at different exposure time, allowing real time recording of the reaction kinetics (one-step reaction). After washing of the arrays, fluorescence was detected again at different exposure times.

### Signal Quantification:

The fluorescent signal intensity for each peptide was analyzed using BioNavigator 6.3 software (PamGene International BV, 's-Hertogenbosch, The Netherlands) a statistical analysis and visualization software tool (https://www.pamgene.com/en/bionavigator.htm). Around each spot a local background was calculated. This value was subtracted from the signal intensity, resulting in signal minus background (SigmBg). For signal quantification, at each time point the slope of the SigmBg versus exposure times was calculated in order to increase the dynamic range.

### Calculation of IC50 Values:

IC50 values were calculated in Graphpad PRISM software (Version 8.4.2, San Diago, CA, USA), using the after wash integrated relative signal intensities of each compound in comparison to DMSO control. Nonlinear regression curve fitting model was used on relative signal intensity for each peptide to get the inhibitor-response graph and IC50 values.

### EXAMPLES

The following Examples are detailed by way of illustration only and are not to be construed as limiting in spirit or in scope, many modifications both in materials and in methods will be apparent to those skilled in the art.

### Example 1

Excess amount of a kinase inhibitor was added to water containing various amounts of γ-cyclodextrin. The suspensions formed were placed in an ultrasonic bath where they were sonicated at 30° C. for 30 min. After cooling to room temperature (22-23° C.) the vials were opened and small amount of the pure drug added to the media to promote drug precipitation and then equilibrated in a shaker (KS 15 A Shaker, EB Edmund Bühler GmbH, Germany) at room temperature under constant agitation for 7 days. Finally, the suspensions were centrifuged at 12000 rpm for 15 min (Heraeus Pico 17 Centrifuge, Thermo Fisher Scientific, Germany), the supernatants diluted with Milli-Q water and analyzed by HPLC.

Phase-solubility analysis was performed according to method described by Higuchi and Connors (T. Higuchi, KA Connors: Phase-solubility techniques, Adv. Anal. Chem. Instrum. 4, 117-212, 1965). The complexation efficiency (CE) (Eq.5) was calculated from slope of the initial linear portion of the drug concentration against γ-cyclodextrin (γCD) concentration profiles assuming drug/γ-cyclodextrin 1:1 complex formation (i.e. that the molar ration of the kinase inhibitor and γ-cyclodextrin in the complex is one-to-one). The results are shown in Table 1. The CE ranges from 0.0578 for cediranib to 0.00002 for pazopanib and regorafenib. It is observed that although cediranib has lower S₀ it has higher CE than dovitinib. Same is true for acrizanib and axitinib.

TABLE 1
The calculated solubilities (S) of some kinase inhibitors in
pure water at 25° C., their complexation efficacy with the
natural γ-cyclodextrin (γCD) in aqueous γ-cyclodextrin
solution saturated with a given kinase inhibitor.

| Kinase | | S in water | |
|---|---|---|---|
| inhibitor | MW (Da) | (mg/mL) ^{a} | CE |
| Acrizanib | 445.40 | 0.001 | 0.0004 |
| Axitinib | 386.47 | 0.04 | 0.0002 |
| Cediranib | 450.51 | 0.2 | 0.0578 |
| Dovitinib | 392.43 | 0.4 | 0.0110 |
| Dovitinib dilactate | 572.59 | 0.3 ^{b} | 0.159 |
| Motesanib | 373.45 | 0.01 | 0.0052 |
| Pazopanib | 437.52 | 0.002 | 0.00002 |
| Regorafenib | 482.82 | 0.0001 | 0.00002 |

^{a} Calculated solubility at pH 7 and 25° C. (ACS, 2020).
^{b} Experimental solubility at pH 6.5.

### Example 2

Six VEGFR2 inhibitors (i.e. axitinib, linifanib, cabozantinib, anlotinib, orantinib and nintedanib) were selected and their Tyrosine PamChip-based kinase activity profiling on PamGene's Tyrosine (phosphotyrosine kinase; PTK) arrays to confirm inhibitor specificity for VEGFR2 over EGFR (ParmGene Int BV, Shertogenbosch, Netherlands). The selected VEGFR2 inhibitors and a specific EGFR inhibitor (as control) were tested in cornea and retina tissue lysates from rabbits. For optimization, 6 different concentrations of each inhibitor (spanning a 100,000-fold range) were tested against untreated lysate of one cornea and one retina. Subsequently, one selected concentration of each inhibitor was tested for the 3 biological replicates of cornea and retina. A total of 10-12 PTK runs (each run consisted of 12 arrays). The results are shown in Table 2:

TABLE 2
The experimental IC₅₀ (nM) values for VEGFR2 and EGFR of
six kinase inhibitors and the EGFR/VEGFR2 IC50 ratio.

| | | | EGFR/ | VEGFR2 | |
|---|---|---|---|---|---|
| | | | VEGFR2 | inhibition at | |
| Kinase | IC50 (nM) | | IC50 | 1 mM (%) | |
| inhibitor | VEGFR2 | EGFR | ratio | Cornea | Retina |
| Cabozanitinib | 12 | >100,000 | >8333 | 90.52 | 74.82 |
| Axitinib | 14 | >100,000 | >7142 | 58.70 | 41.37 |
| Nintedanib | 9 | ~50,000 | ~5555 | 95.92 | 94.33 |

In the recombinant assays, axitinib and cabozantinib are potent inhibitors for VEGFR that do not inhibit EGFR. In cornea, nintedanib is most potent VEGFR2 inhibitor, followed by cabozantinib and axitinib. In retina, nintedanib remains most potent VEGFR2 inhibitor and other inhibitors show similar potency. Results of the assay are graphically presented in FIG. 1.

Most importantly, the results show that the preferred kinase inhibitors inhibit VEGFR2, but only to a very limited extent EGFR, so that side effects are minimized.

### Example 3

Table 3 shows five ophthalmic formulations containing dovitinib free base or dovitinib lactate. The components were suspended in water and the formed suspension heated in an autoclave at 121° C. for 20 minutes. Then the suspensions were equilibrated at 22-23° C. for 7 days under constant agitation. During the equilibration, the samples were adjusted to a pH 6.5±0.1 with aqueous 0.10 N hydrochloric acid (HCl) solution or aqueous 1.0 N sodium hydroxide (NaOH) solution and the volume adjusted with purified water. After equilibrium was attained, the suspensions were analyzed for dovitinib, both before (i.e. the total dovitinib concentration) and after filtration through 0.45 mm membrane filter (i.e. the dissolved dovitinib concentration), by HPLC. When the free base was used, the amount of dovitinib that could be included in the γ-cyclodextrin aggregates was relatively low or 0.3% (w/v).

The use of dovitinib lactate resulted in a surprisingly significant increase of drug that could be dissolved and suspended, respectively. Further significant enhancement of drug dissolution/suspension was observed by addition of EDTA as a chelating agent and surface active polymers like tyloxapol. As can be seen from Table 4, an almost 10-fold increase was achieved.

The solid fraction was calculated from the concentration of dovitinib before and after filtration. About 60 to 75% of dovitinib was in solid dovitinib/γ-cyclodextrin complex microparticles with a mean diameter (D₅₀) of less than 10 μm and 25 to 40% of the drug was dissolved as free drug, drug/γ-cyclodextrin complexes or dissolved dovitinib/γ-cyclodextrin complex nanoparticles with diameter between 60 and 130 nm. The particle sizes were determined by dynamic light scattering and transmission electron microscope.

TABLE 3
Dovitinib eye drop formulations.

| | Concentration (% w/v) | | | | |
|---|---|---|---|---|---|
| Ingredient | DF1 | DF2 | DF3 | DF4 | DF5 |
| Dovitinib | 0.3 | 0.3 | | | 2.45 |
| Dovitinib lactate | | | 1.5 | 3.0 | 0.82^{a} |
| γ-Cyclodextrin | 10 | 10 | 15.0 | 15.0 | 20 |
| Poly(vinyl alcohol) | | 0.25 | | | |
| Hydroxypropyl methylcellulose | 0.25 | 0.25 | | | |
| Poloxamer 407 | 0.20 | 0.20 | 0.20 | 0.20 | 0.20 |
| Tyloxapol | | 0.10 | 0.10 | 0.10 | |
| Disodium edetate (EDTA) | 0.10 | 0.10 | 0.10 | 0.10 | 0.10 |
| NaCl | 0.58 | 0.58 | 0.55 | 0.0 | 0.13 |
| 0.1N HCl/1N NaOH | pH 6.0 | pH 6.0 | pH 6.0 | pH 6.0 | pH 6.0 |
| Purified water | ad 100 ml | ad 100 ml | ad 100 ml | ad 100 ml | ad 100 ml |

^{a}0.82% (w/v) dovitinib dilactate (572.6 g/mol) corresponds to 0.56% (w/v) dovitinib base (392.4 g/mol).

### Example 4

In order to keep high concentrations of dissolved kinase inhibitors in the aqueous tear fluid the dissolution of the kinase inhibitor must proceed very quickly upon media dilution. The test formulation was above DF3 and the reference formulation was AF1 comprising acrizanib (Table 4 below). A dissolution test was performed by direct adding of a formulation aliquot into defined volume of water under constant stirring speed. The formulation/water ratio (final dilution) selection was based on the quantification limit of the used HPLC method and on the acrizanib solubility. The final dilution of 450 times was selected. At certain time after adding the formulation to water a sample of about 1 ml was taken from a stirring media, filtered through 0.45 μm filter and transferred to an HPLC vial for analysis.

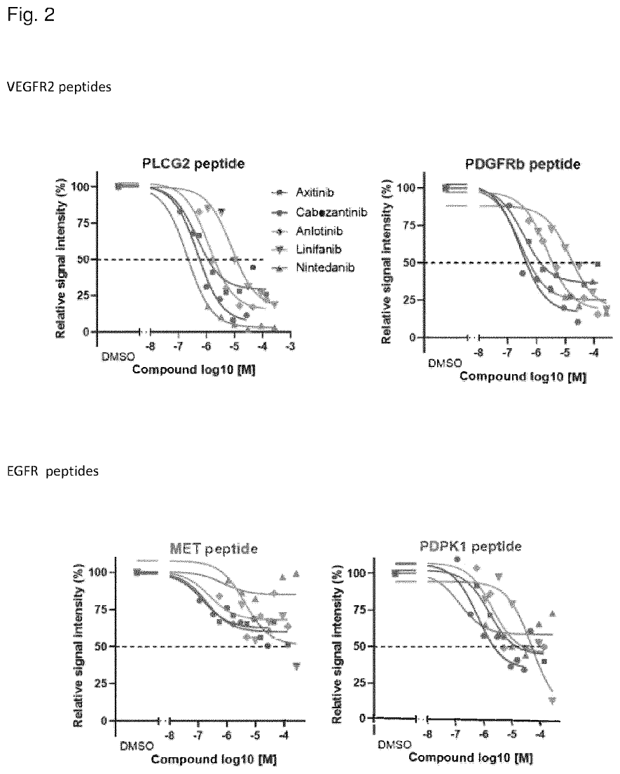

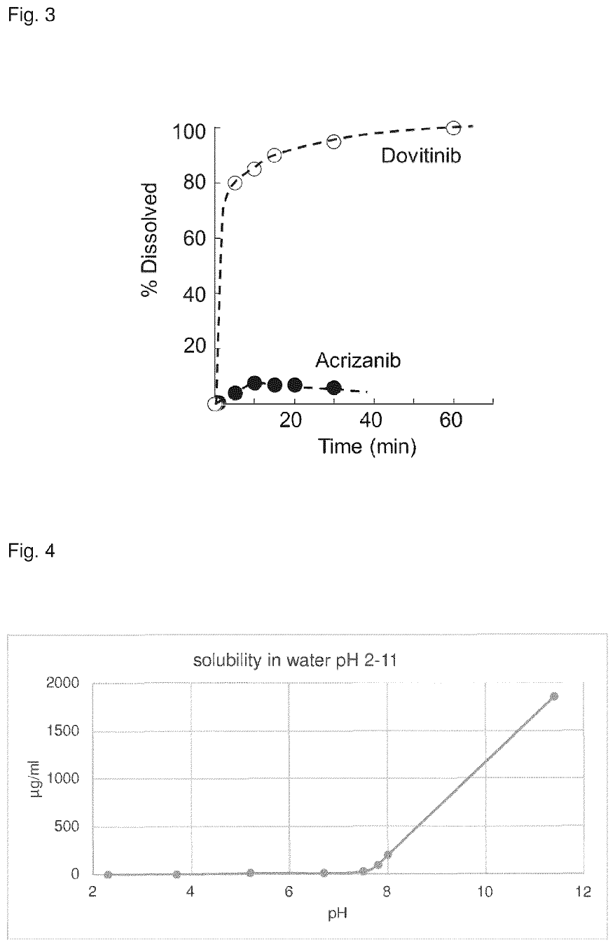

As can be seen from FIG. 2 only about 7.5% of acrizanib dissolves with a maximum dissolution at about 10 minutes, while about 80% of dovitinib dissolves within the first 5 minutes and 100% has dissolved within one hour. The slower terminal dovitinib dissolution is due to saturation of the aqueous dissolution media. Moreover, the dissolution test shows that after 10 minutes the concentration of acrizanib decreases, which can be related to instability of the formed acrizanib/γ-cyclodextrin complex and precipitation of free acrizanib. Indeed, the formation of large insoluble particles (flakes) was observed in stirring media at 10 to 30 minutes. This was confirmed by laser scattering data. The complexation efficacy of acrizanib was determined to be 0.0004 while that of dovitinib is 0.011 (Table 1). The fast dissolution of the solid dovitinib is essential for effective topical delivery of the drug into the eye.

TABLE 4
Composition of the acrizanib hydrochloride
reference eye drops (AF1).

| Component | AF1 |
|---|---|
| Acrizanib hydrochloride | 2.0 |
| γ-Cyclodextrin | 6.0 |
| EDTA | 0.1 |
| NaOH/HCl q.s. to | pH 5.7 |
| Purified water q.s. to | 100 ml |

### Example 5

Table 5 provides three ophthalmic formulations containing cediranib maleate. Cediranib maleate possesses significant greater solubility than the free base and gives higher complexation efficacy. Further improvement of the complexation efficacy is obtained by addition of EDTA and polymers like tyloxapol. Sufficient cediranib solubility and complexation efficacy with γ-cyclodextrin was obtained through combination of salts, chelating agents and surface active agents, so that the considerably more pharmaceutical active ingredient could be dissolved/suspended as compared to using the free base.

The solid fraction was determined as described in Example 3. About 87% of cediranib was in solid cediranib/γ-cyclodextrin complex microparticles with diameter of less than 10 μm and about 13% of the drug was dissolved as free drug, drug/γ-cyclodextrin complexes or dissolved cediranib/γ-cyclodextrin complex nanoparticles with diameter below 200 nm.

TABLE 5
Composition of cediranib maleate eye drops.

| | Concentration (% w/v) | | |
|---|---|---|---|
| Ingredient | CF1 | CF2 | CF3 |
| Cediranib maleate | 1.5 | 3.0 | 3.0 |
| Y-Cyclodextrin | 15.0 | 15.0 | 20.0 |
| Poloxamer 407 | 0.2 | 0.2 | |
| Tyloxapol | 0.1 | 0.1 | |
| Polyethylene | | | 0.20 |
| glycol 400 | | | |
| EDTA | 0.1 | 0.1 | 0.05 |
| NaCl | 0.55 | 0.0 | 0.13 |
| 0.1N HCI/ | pH 6.0 | pH 6.0 | pH 6.0 |
| 1N NaOH | | | |
| Purified water | ad 100 ml | ad 100 ml | ad 100 ml |

### Example 6

Table 6 below provides a listing of ingredients suitable for other exemplary ophthalmic formulations of the above cediranib aqueous suspension of the present invention and desired weight/volume percentages for those ingredients. The chemical stability was evaluated by determining the cediranib concentration in the formulations before and after autoclaving at 121° C. for 20 minutes.

TABLE 6
Composition of cediranib maleate eye drops.

| | Concentration (% w/v) | | |
|---|---|---|---|
| Ingredient | CF4 | CF5 | CF6 |
| Cediranib maleate | 3.0 | 3.0 | 3.0 |
| Y-Cyclodextrin | 15.0 | 15.0 | 15.0 |
| Propylene glycol | 0.0 | 0.0 | 0.1 |
| Glycerol | 0.0 | 0.0 | 0.1 |
| Poly(vinyl alcohol), | 0.0 | 0.0 | 0.2 |
| 30-70k | | | |
| Hydroxypropyl | 0.0 | 0.0 | 0.1 |
| methylcellulose | | | |
| Poloxamer 407 | 0.0 | 0.0 | 0.1 |
| EDTA | 0.1 | 0.1 | 0.1 |
| Riboflavin | 0.0 | 0.1 | 0.1 |
| L-Arginin | 0.0 | 0.0 | 0.1 |
| NaCl | 0.5 | 0.5 | 0.5 |
| NaOH/HCI q.s. to | pH 5 | pH 5 | pH 5 |
| Purified water | 100 ml | 100 ml | 100 ml |
| q.s. to | | | |
| Solid drug | 69.1 | 72.7 | 72.5 |
| fraction (%) | | | |
| Fraction | 17.01 | 9.98 | 7.69 |
| degraded (%) | | | |

The aqueous eye drop microsuspensions containing riboflavin and polymers did retard or prevent the drug loss during heating process. The stability of formulation CF6 was investigated and the results shown in Table 7.

TABLE 7
Drug assay of CF6 during storage for 3 months.

| | Drug concentration (mg/ml) | | Drug concentration (%) | |
|---|---|---|---|---|
| Months | Mean | SD | Mean | SD |
| 5º C. | | | | |
| 0 | 30.56 | 1.54 | 101.87 | 5.12 |
| 1 | 30.46 | 1.59 | 101.54 | 5.30 |
| 2 | 31.24 | 0.24 | 104.12 | 0.81 |
| 3 | 30.74 | 1.46 | 102.47 | 4.87 |
| 25° C | | | | |
| 0 | 31.18 | 0.57 | 103.91 | 1.86 |
| 1 | 30.30 | 0.06 | 100.98 | 0.16 |
| 2 | 30.25 | 0.19 | 100.81 | 0.59 |
| 3 | 30.05 | 0.05 | 100.15 | 0.20 |
| 40º C | | | | |
| 0 | 30.84 | 0.40 | 102.81 | 1.35 |
| 1 | 29.26 | 0.11 | 97.52 | 0.35 |
| 2 | 27.02 | 0.16 | 90.06 | 0.53 |
| 3 | 26.28 | 0.37 | 87.59 | 1.22 |

### Example 7

The above dovitinib salt formulation DF5 and the above cediranib salt formulation CF3 were tested in rabbits, 8 rabbits for each drug, 4 rabbits at each time point. One eye drop (50 μl) was administered to the left eye and the levels of the drug measured at 2 hours and 6 hours after administration. The drug concentrations were measured in the cornea, aqueous humor, sclera, retina and vitreous humor. All ocular tissue samples were homogenized using a Precellys Evolution bead homogenizer with an acetonitrile/methanol mixture as homogenization solvent in ratio 1:4 (4 μL solvent for each mg ocular tissue). Homogenates were centrifugated and supernatant was further diluted prior to sample analysis. A reversed phase LC-MS/MS methods were developed and qualified with a standard range 1-1000 μM in surrogate matrix. The dovitinib and cediranib concentrations in the left eye (i.e. the study eye) are shown in Tables 8 and 9, respectively.

TABLE 8

| Concentration of dovitinib (in picomoles | | |
|---|---|---|
| per gram; pmol/g) in various | | |
| eye tissues at 2 and 6 hours after topical | | |
| administration of one drop of aqueous | | |
| 3.0% dovitinib/y-cyclodextrin eye drop microsuspension. | | |
| | Concentration (pmol/g) | |
| Tissue | 2 hours | 6 hours |
| Cornea | 134,000 ± 41,200 | 91,400 ± 50,800 |
| Aqueous humor | 8.75 ± 3.68 | 6.67 ± 0.64 |
| Sclera | 11,300 ± 3,170 | 3,870 ± 2,580 |
| Retina | 79.4 ± 14.5 | 63.8 ± 16.2 |
| Vitreous humor | 4.76 ± 1.15 | 3.27 ± 3.08 |
| Cornea/retina ratio | 1690 | 1430 |

TABLE 9
Concentration of cediranib (in nanograms
per gram; ng/g) in various
eye tissues at 2 and 6 hours after topical
administration of one drop of aqueous
3.0% cediranib/y-cyclodextrin eye drop microsuspension.

| | Concentration (ng/g) | |
|---|---|---|
| Tissue | 2 hours | 6 hours |
| Cornea | 361,000 ± 0000 | 114,000 ± 27,500 |
| Aqueous humor | 1,990 ± 1880 | 64.9 ± 18.3 |
| Sclera | 25,000 ± 8,050 | 4,430 ± 1,250 |
| Retina | 511 ± 23.3 | 169 ± 16.7 |
| Vitreous humor | 27.0 ± 13.5 | 5.00 ± 2.01 |
| Cornea/retina ratio | 706 | 675 |

The results show that the concentration in the cornea is from 675 times to 1690 times higher than in the retina. When applied topically the cornea is more accessible to the kinase inhibitors than the retina and, thus, the corneal drug concentration will always be much higher than the retinal concentration. The ocular toxicity of kinase inhibitors is mainly associated with the eye surface and the anterior section, and especially with the EGFR in the cornea, while the therapeutic effect is associated with the posterior section, especially with the VEGFR2 in the retina. The kinase inhibitors have to have over 2000-fold higher, and preferable over 5000-fold higher, affinity for VEGFR2 than for EGFR to be safely administered topically to the eye in aqueous eye drops.

### Example 8

Reference aqueous dovitinib (free base) and cediranib (free base) eye drop microsuspensions were prepared and tested in rabbits as described in Example 7. The 3.0% (w/v) dovitinib reference eye drops contained tyloxapol (0.3% w/v) and sodium chloride (0.8% w/v) in purified water. The pH of the eye drops was 5.8, the osmolarity was 290 mOsm/kg and the mean particle size was 6 μm. Only 1.3% of dovitinib was in solution. The 3.0% (w/v) cediranib reference eye drops contained tyloxapol (0.3% w/v) and sodium chloride (0.8% w/v) in purified water. The pH of the eye drops was 5.9, the osmolarity was 269 mOsm/kg and the mean particle size 2 μm. Only 1% of cediranib was in solution. One eye drop (50 μl) was administered to the left eye and the levels of the drug measured at 2 hours and 6 hours after administration. The dovitinib and cediranib concentrations in the left eye (i.e. the study eye) are shown in Tables 10 and 11, respectively.

TABLE 10

| Concentration of dovitinib (in nanogram per | | |
|---|---|---|
| gram; ng/g) in various | | |
| eye tissues at 2 and 6 hours after topical | | |
| administration of one drop of aqueous | | |
| 3.0% dovitinib reference eye drop | | |
| microsuspension. | | |
| | Concentration (ng/g) | |
| Tissue | 2 hours | 6 hours |
| Cornea | 1.474 ± 744 | 1,425 ± 1,356 |
| Aqueous humor | 0.90 ± 0.11 | 0.55 ± 0.19 |
| Sclera | 172 ± 33.6 | 110 ± 63.5 |
| Retina | 22.2 ± 5.7 | 32.5 ± 8.7 |
| Vitreous humor | 1.38 ± 0.00 | 0.23 ± 0.00 |
| Cornea/retina ratio | 66 | 44 |

TABLE 11
Concentration of cediranib (in picomoles per
gram; pmol/g) in various
eye tissues at 2 and 6 hours after topical
administration of one drop of aqueous
3.0% cediranib reference eye drop microsuspension.

| | Concentration (pmol/g) | |
|---|---|---|
| Tissue | 2 hours | 6 hours |
| Cornea | 121,382 ± 42,951 | 60,146 ± 24,284 |
| Aqueous humor | 62.0 ± 18.3 | 43.0 ± 5.7 |
| Sclera | 7,394 ± 1,512 | 3,008 ± 2,089 |
| Retina | 202 ± 48.3 | 163 ± 32.8 |
| Vitreous humor | 7,63 ± 5,18 | 4.82 ± 1.65 |
| Cornea/retina ratio | 601 | 369 |

Comparing the drug concentrations in the tissue obtained on use of the active pharmaceutical ingredient in salt form with the use of the free base, it is evident that significantly more drug migrates by passive diffusion into membranes and to the target tissue (retina) on use of the drug in salt form.

### Example 9

TABLE 12

| Compositions comprising | |
|---|---|
| nintedanib salts | |
| Ingredient | % w/V |
| Nintedanib acetate, | 0.5 or 1.0 or |
| lactate, maleate, | 2.0 or 3.0 |
| esylate or aspartate | |
| Y-Cyclodextrin | 5 to 30 |
| Poloxamer | 0 to 2 |
| Tyloxapol | 0 to 2 |
| Polyethylene glycol | 0 to 2 |
| Polyvinylalcohol | 0 to 2 |
| Hydroxypropyl methylcellulose | 0 to 2 |
| Polyvinylpyrrolidone | 0 to 5 |
| Carboxymethyl cellulose | 0 to 2 |
| Hexadimethrine bromide | 0 to 2 |
| EDTA | 0 to 1 |
| Sodium chloride | 0 to 0.8 |
| Sodium hydroxide/ | Sufficient to |
| hydrochloric acid | achieve targeted |
| | pH of 4 to 7.5 |
| Purified water | ad 100 ml |

TABLE 13
Compositions comprising cabozantinib salts

| Ingredient | % w/v |
|---|---|
| Cabozantinib acetate, | 0.5 or 1.0 or |
| lactate, malate, | 2.0 or 3.0 |
| chloride or aspartate | |
| Y-Cyclodextrin | 5 to 30 |
| Poloxamer | 0 to 2 |
| Tyloxapol | 0 to 2 |
| Polyethylene glycol | 0 to 2 |
| Polyvinylalcohol | 0 to 2 |
| Hydroxypropyl methylcellulose | 0 to 2 |
| Polyvinylpyrrolidone | 0 to 5 |
| Carboxymethyl cellulose | 0 to 2 |
| Hexadimethrine bromide | 0 to 2 |
| EDTA | 0 to 1 |
| Sodium chloride | 0 to 0.8 |
| Sodium hydroxide/ | Sufficient to |
| hydrochloric acid | achieve targeted |
| | pH of 4 to 7.5 |
| Purified water | ad 100 ml |

TABLE 14
Compositions comprising axitinib salts

| Ingredient | % w/V |
|---|---|
| Axitinib acetate, | 0.5 or 1.0 or |
| lactate, maleate or | 2.0 or 3.0 |
| aspartate | |
| Y-Cyclodextrin | 5 to 30 |
| Poloxamer | 0 to 2 |
| Tyloxapol | 0 to 2 |
| Polyethylene glycol | 0 to 2 |
| Polyvinylalcohol | 0 to 2 |
| Hydroxypropyl methylcellulose | 0 to 2 |
| Polyvinylpyrrolidone | 0 to 5 |
| Carboxymethyl cellulose | 0 to 2 |
| Hexadimethrine bromide | 0 to 2 |
| EDTA | 0 to 1 |
| Sodium chloride | 0 to 0.8 |
| Sodium hydroxide/ | Sufficient to |
| hydrochloric acid | achieve targeted |
| | pH of 4 to 7.5 |
| Purified water | ad 100 ml |

TABLE 15
Composition of axitinib eye drops

| Ingredient | % w/v |
|---|---|
| axitinib free base | 1 |
| y-CD | 6 |
| EDTA | 0.1 |
| NaCI | 0.5 |
| poloxamer | 0.5 |
| tyloxapol | 1.0 |
| NaOH/maleic acid | to pH 5 |
| Water | ad 100% |

TABLE 16
Composition of orantinib eye drops

| Ingredient | % w/V |
|---|---|
| orantinib free acid | 1 |
| y-CD | 12 |
| EDTA | 0.1 |
| NaCI | 0.5 |
| poloxamer | 0.5 |
| NaOH/HCI | to pH 7.4 |
| Water | ad 100% |

### Example 10

The phase solubility profile of orantinib free acid was determined in water at pH 2-11. The phase solubility profile is given in FIG. 4.

### Example 11

Stability of orantinib in an autoclave was determined by mixing orantinib (free acid), MQ water and NaOH in glass vials, shake them for three days, followed by filtering through a filter of a pore size of 0.45 micrometer. Each vial was then split up into 2 vials. One vial of each set was autoclaved and all samples analyzed by HPLC. The results are given in the table below.

Samples of orantinib in MQ water,

| | | before | after | |
|---|---|---|---|---|
| | | autoclave | autoclave | |
| sample | pH | (μg/ml) | (μg/ml) | Recovery |
| 1-29 | 7.4 | 60 | 64 | 107% |
| 2-29 | 7.6 | 182 | 176 | 96% |
| 3-29 | 7.8 | 321 | 316 | 98% |

### Example 12

Phase solubility screening with gamma-cyclodextrin at pH 6. Excess amount of orantinib free acid was added to water containing various amounts of γ-cyclodextrin. The suspensions formed were autoclaved for 15 minutes at 121° C. After cooling to room temperature (22-23° C.) the vials were opened and small amount of the pure drug added to the media to promote drug precipitation and then equilibrated in a shaker (KS 15 A Shaker, EB Edmund Bühler GmbH, Germany) at room temperature under constant agitation for 4 hours. Finally, the suspensions were centrifuged at 12000 rpm for 15 min (Heraeus Pico 17 Centrifuge, Thermo Fisher Scientific, Germany), the supernatants diluted with Milli-Q water and analyzed by HPLC.

| % | | Orantinib |
|---|---|---|
| (w/v) | | solubility |
| yCD | pH | (μg/ml) |
| 0 | 6.3 | 4 |
| 5 | 6.1 | 14 |
| 10 | 6.2 | 17 |
| 15 | 6.0 | 12 |

### Example 13

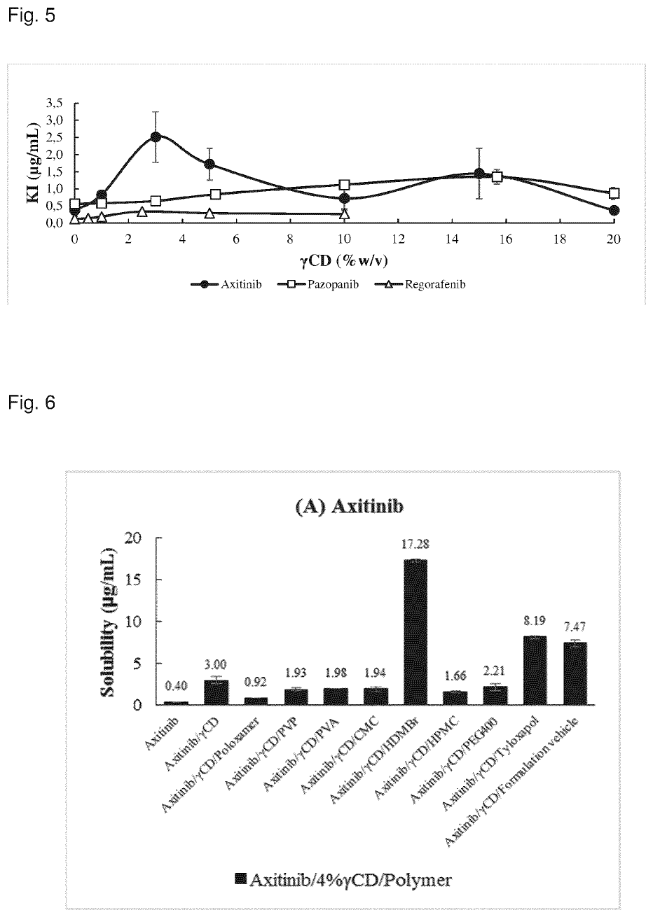

Phase solubility study of axitinib. The free bases of axitinib, pazopanib and regorafenib were mixed with MQ water and gamma-cyclodextrin in various concentrations. The solubility was determined in dependence of % w/v of gamma-cyclodextrin. Results are given in FIG. 5.

The solubility of the axitinib free base was determined in admixture with gamma-cyclodextrin (γCD) and various polymers. Results are given in FIG. 6, wherein HDMBR is hexadimethrine bromide (>94% pure by titration with molecular weight 374 kDa) and the formulation vehicle consists of 0.1% (w/v) EDTA, 0.02% (w/v) benzalkonium chloride, and 0.05% (w/v) sodium chloride in pure water.

### Example 14

The axitinib free base was mixed with various acids to determine its solubility. 10 mg/ml of axitinib and equal molar ratio of various acids was added to water containing 50 mg/ml of γ-cyclodextrin (γCD). The suspensions formed was kept on a shaker (KS 15 A Shaker, EB Edmund Bühler GmbH, Germany) at room temperature under constant agitation for 3 days. Finally, the suspensions were centrifuged at 12000 rpm for 15 min (Heraeus Pico 17 Centrifuge, Thermo Fisher Scientific, Germany), the supernatants diluted with 50% acetonitrile and analyzed by HPLC. Results are below.

| | | 10 mg/ml Axi + 50 mg/ml yCD + | | |
|---|---|---|---|---|
| | | acid (1:1 molar vs Axi) | | |
| | | | Axi in | YCD in |
| | | | solution | solution |
| Acid | pKa | pH | mg/ml | mg/ml |
| Acetic | 4.76 | 3.5 | 0.011 | 24 |
| Formic | 3.75 | 2.8 | 0.055 | 21 |
| Lactic | 3.86 | 2.8 | 0.066 | 24 |
| Ascorbic | 4.17 | 3.1 | 0.032 | 27 |
| Malic | 3.51, 5.03 | 3.0 | 0.034 | 24 |
| Tartaric | 2.98, 4.34 | 2.7 | 0.071 | 22 |
| Maleic | 1.94, 4.34 | 2.3 | 0.169 | 25 |
| Hydrochloric | -5.9 | 3.2 | 0.005 | 21 |
| None | | 9.1 | 0.001 | 22 |

### EMBODIMENTS

Item 1. An aqueous composition comprising drug/cyclodextrin complexes of:
a tyrosine kinase inhibitor or a salt thereof, and
a cyclodextrin
wherein said complexes have a complexation efficacy (CE) of more than 0.01, preferably more than 0.1 in the aqueous composition, and/or
the tyrosine kinase inhibitor or a salt thereof has a ratio of the half maximal inhibitory concentration (IC₅₀) of the epidermal growth factor receptors (EGFR) to the half maximal inhibitory concentration (IC₅₀) of the vascular endothelial growth factor receptors (VEGFR2) that is greater than 2000, preferably greater than 5000.

Item 2. An aqueous composition according to item 1, wherein the tyrosine kinase inhibitor has a pKa of 2 to 8.

Item 3. The aqueous composition according to item 1 or 2, wherein the tyrosine kinase inhibitor or a salt thereof is selected from the group of anlotinib, axitinib, cabozantinib, foretinib, linifanib, nintedanib, orantinib, ZM323881, preferably axitinib, orantinib and nintedanib.

Item 4. The aqueous composition according to any one of items 1 to 3, wherein the tyrosine kinase inhibitor or a salt thereof is selected from axitinib, and nintedanib.

Item 5. The aqueous composition according to any of items 1 to 4, comprising a salt of said tyrosine kinase inhibitor selected from the group of acetate, chlorate, esylate, lactate, malate, maleate, aspartate.

Item 6. The aqueous composition according to any of items 1 to 4, wherein the tyrosine kinase inhibitor or a salt thereof is orantinib.

Item 7. The aqueous composition according to item 6, wherein a salt of said tyrosine kinase inhibitor is sodium or potassium.

Item 8. The aqueous composition according to any of items 1 to 7, wherein said cyclodextrin is γ-cyclodextrin.

Item 9. The aqueous composition according to any of items 1 to 8, further comprising 0.1% (w/v) to 5% (w/v) of a chelating agent as a stabilizer.

Item 10. The aqueous composition according to item 9, wherein the chelating agent is a divalent or polyvalent carboxylic acid.

Item 11. The aqueous composition according to item 10, wherein the chelating agent is selected from the group of ethylenediamine-tetraacetic acid (EDTA), 2,2′,2″-nitrilotriacetic acid (NTA), malic acid, maleic acid, succinic acid, and citric acid.

Item 12. The aqueous composition according to any of items 1 to 11, which is a microsuspension comprising particles of said complexes cyclodextrin and tyrosine kinase inhibitor, wherein from about 5% (w/v) to about 50% (w/v) of the tyrosine kinase inhibitor is in solution, as dissolved free drug or as dissolved drug/cyclodextrin complex(es), and from about 50% (w/v) to about 95% (w/v) of the tyrosine kinase inhibitors is in solid drug/cyclodextrin complex particles.

Item 13. The aqueous composition according to any of items 1 to 12, which is a microsuspension comprising particles of said complexes cyclodextrin and tyrosine kinase inhibitor, and the average size D₅₀ of the particles in the solid phase is from about 0.1 μm to about 500 μm, typically from 1 μm to 50 μm,

Item 14. The aqueous composition according to any of items 1 to 13, wherein the composition comprises from about 0.25% to about 40% (w/v) of cyclodextrin, typically γ-cyclodextrin.

Item 15. The aqueous composition according to any of items 1 to 14, wherein the composition comprises from about 0.1 to 5% (w/v) of surface active polymer.

Item 16. The aqueous composition according to any of items 1 to 15, further comprising one or more surface active polymers selected from the group of poloxamer, tyloxapol, polyalkyleneglycol, hydroxyalkylcellulose, hydroxyalkyl alkylcellulose, and polyvinyl alcohol are present.

Item 17. The aqueous composition according to any of items 1 to 16, further comprising a tonicity adjusting agent.

Item 18. The aqueous composition according to item 17, wherein the tonicity adjusting agent comprises sodium chloride.

Item 19. The aqueous composition according to item 18, wherein the composition comprises 0.01% (w/v) to 0.9% (w/v) of sodium chloride.

Item 20. The aqueous composition according to any of items 1 to 19 for use in the topical treatment of retinal diseases.

Item 21. The aqueous composition according to any of items 1 to 19 for use in treating a condition of posterior segment and/or the anterior segment of the eye.

Item 22. The aqueous composition for use according to item 20, wherein said condition is selected from the group of age-related macular degeneration (AMD), diabetic retinopathy (DR), diabetic macular edema (DME), retinopathy of prematurity and pathologic choroidal neo vascularization (CNV).

Item 23. A method for treating a condition of the posterior segment and/or the anterior segment of the eye in a subject in need thereof, said method comprising applying topically to the eye surface of said subject, an aqueous composition according to any one of items 1 to 19 comprising as a tyrosine kinase inhibitor as the active principle, in an amount which delivers a therapeutically effective amount of said tyrosine kinase inhibitor to said segment or segments of the eye.

We claim:

1. A method of treatment of a posterior ocular disease comprising administering an effective amount of an aqueous ophthalmic composition comprising a tyrosine kinase inhibitor or a salt thereof complexed with γ-cyclodextrin;
wherein the tyrosine kinase inhibitor/γ-cyclodextrin complexes comprise microparticles with a diameter D₅₀ ranging from about 1 μm to 50 μm;
the tyrosine kinase inhibitor/γ-cyclodextrin complexes have a complexation efficacy (CE) of more than 0.01; and
the tyrosine kinase inhibitor or a salt thereof has a ratio of the half maximal inhibitory concentration (IC₅₀) of epidermal growth factor receptors (EGFR) to the half maximal inhibitory concentration (IC₅₀) of vascular endothelial growth factor receptors (VEGFR2) of greater than 2000.

2. The method of claim 1, wherein the posterior ocular disease is a retinal disease.

3. The method of claim 1, wherein the posterior ocular disease is selected from the group consisting of age-related macular degeneration (AMD), diabetic retinopathy (DR), diabetic macular edema (DME), retinopathy of prematurity and pathologic choroidal neo vascularization (CNV).

4. The method of claim 1, wherein the complexation efficacy (CE) is more than 0.1.

5. The method of claim 1, wherein the tyrosine kinase inhibitor or a salt thereof has a ratio of the half maximal inhibitory concentration (IC₅₀) of the epidermal growth factor receptors (EGFR) to the half maximal inhibitory concentration (IC₅₀) of the vascular endothelial growth factor receptors (VEGFR2) of greater than 5,000.

6. The method of claim 1, wherein the tyrosine kinase inhibitor or a salt thereof is selected from the group consisting of anlotinib, axitinib, cabozantinib, cediranib, dovitinib, foretinib, linifanib, nintedanib, orantinib, and ZM323881 or a salt thereof.

7. The method of claim 1, wherein the tyrosine kinase inhibitor or a salt thereof is cediranib, or dovitinib or a salt thereof.

8. The method of claim 1, wherein the tyrosine kinase inhibitor or a salt thereof is axitinib, orantinib or nintedanib or a salt thereof.

9. The method of claim 1, wherein the aqueous ophthalmic composition comprising a salt of the tyrosine kinase inhibitor complexed with γ-cyclodextrin, wherein the salt is selected from the group consisting of acetate, chlorate, esylate, lactate, malate, maleate, aspartate, sodium, and potassium.

10. The method of claim 1, wherein the aqueous ophthalmic composition comprises a mixture of dovitinib and dovitinib lactate complexed with γ-cyclodextrin.

11. The method of claim 1, wherein the aqueous ophthalmic composition comprises cediranib maleate complexed with γ-cyclodextrin.

12. The method of claim 1, wherein the aqueous ophthalmic composition comprises from about 0.25% by weight based on the volume of the composition (w/v) to about 40% (w/v) of the γ-cyclodextrin.

13. The method of claim 1, wherein the tyrosine kinase inhibitor/γ-cyclodextrin complexes comprise microparticles with a diameter D₅₀ ranging from about 1 μm to about 10 μm.

14. The method of claim 1, wherein the aqueous ophthalmic composition further comprises from about 0.1% by weight based on the volume of the composition (w/v) to about 5% (w/v) of a chelating agent.

15. The method of claim 14, wherein the chelating agent is selected from the group consisting of ethylenediamine-tetraacetic acid (EDTA), 2,2′,2″-nitrilotriacetic acid (NTA), malic acid, maleic acid, succinic acid, and citric acid.

16. The method of claim 1, wherein the aqueous ophthalmic composition further comprises from about 0.1% by weight based on the volume of the composition (w/v) to about 5% (w/v) of a surface active polymer.

17. The method of claim 16, wherein the surface active polymer is selected from the group consisting of a poloxamer, a tyloxapol, a polyalkyleneglycol, a hydroxyalkyl cellulose, a hydroxyalkyl alkylcellulose, and a polyvinyl alcohol.

18. The method of claim 1, wherein from about 50% by weight based on the volume of the composition (w/v) to about 95% (w/v) of the tyrosine kinase inhibitor or a salt thereof is in a form of the tyrosine kinase inhibitor/γ-cyclodextrin complexes.

19. The method of claim 1, wherein the aqueous ophthalmic composition further comprising a tonicity adjusting agent.

20. The method claim 19, wherein the tonicity adjusting agent comprises sodium chloride.
